(19) United States
(12) Patent Application Publication
(10) Pub. No.: US 2008/0219934 A1
(43) Pub. Date: Sep. 11, 2008

(54) Anionic, Ampholytic Copolymers for Low-Voc-Compositions

(75) Inventors: Son Nguyen Kim, Hemsbach (DE); Matthias Laubender, Schifferstadt (DE); Marianna Pierobon, Ludwigshafen (DE); Gabi Winter, Shanghai (CN)

Correspondence Address: CONNOLLY BOVE LODGE & HUTZ LLP, 1875 EYE STREET, N.W., SUITE 1100, WASHINGTON, DC, 20036, US

(73) Assignee: BASF Aktiengesellschaft, Ludwigshafen (DE)

(21) Appl. No.: 12/063,962

(22) PCT Filed: Aug. 4, 2006
(86) PCT No.: PCT/EP2006/065071

§ 371 Date: Feb. 15, 2008

(30) Foreign Application Priority Data
Aug. 19, 2005 (EP) 05107651.1

(51) Int. Cl. A61K 8/04; C08F 118/02; A61Q 5/00

(52) U.S. Cl. 424/47; 526/319

(57) ABSTRACT

The present invention provides cosmetic compositions containing a copolymer obtainable by the copolymerization of at least one methacrylic acid ester, methacrylic acid, acrylic acid, optionally at least one compound containing amino groups and polymerizable by free radical polymerization, and optionally other olefinically unsaturated compounds polymerizable by free radical polymerization. The copolymerization can be carried out in the presence of silicones f) containing polyalkylene oxide.

The present invention provides cosmetic compositions containing a copolymer obtainable by the copolymerization of at least one methacrylic acid ester, methacrylic acid, acrylic acid, optionally at least one compound d) containing amino groups and polymerizable by free radical polymerization, and optionally other olefinically unsaturated compounds polymerizable by free radical polymerization. If the methacrylic acid ester is or contains ethyl methacrylate or no compound d) is copolymerized, the polymerization is carried out in the presence of at least one silicone f) containing polyalkylene oxide.

In cosmetics, polymers with film-forming properties are used inter alia for strengthening, structural improvement and shaping of the hair. They serve e.g. as conditioners for improving the ease of dry and wet combing, the feel, the luster and the appearance, and for conferring antistatic properties on the hair. Demands made on film-forming polymers for use as strengthening resins are e.g. a good strengthening action, including at high humidity, high flexural rigidity and elasticity, ease of rinsing out of the hair, compatibility in the formulation and a pleasant feel of the treated hair. It is often difficult to provide products with a complex property profile. Thus there is a need for film-forming polymers for hair cosmetics which are capable of forming substantially smooth, non-sticky films, have in particular a good strengthening action and simultaneously confer on the hair good sensory properties such as elasticity, a pleasant feel and volume.

Further desirable properties in hairspray formulations are a good propellant gas compatibility, suitability for use in low-VOC formulations (VOC=Volatile Organic Compounds), a good sprayability, a good solubility in water or aqueous-alcoholic solvent mixtures and a good ease of rinsing. Stricter environmental conditions and a growing ecological awareness are demanding ever-smaller proportions of volatile organic components in cosmetic aerosol compositions such as aerosol hairsprays.

The VOC emission has to be reduced because of considerations relating to environmental problems. For example, in the US State of California, CARB (Californian Air Resources Board), in the Clean Air Act Amendment, controls the maximum permissible content of VOC in various consumer products (e.g. insect sprays, car cleaning products, cosmetics).

The term VOC is known to those skilled in the art. VOC are organic chemical compounds that boil in a range up to approx. 260° C. at normal pressure and can thus enter the atmosphere in gaseous form. Volatile organic compounds include numerous solvents and propellants.

Classification of organic compounds (indoor)
(according to: WHO—World Health Organization 1989)

| | | Boiling range |
|---|---|---|
| Compounds | Abbreviation | [° Celsius] |
| very volatile organic compounds | VVOC | <0 to 50° C. |
| volatile organic compounds | VOC | 50 to 250° C. |
| semivolatile organic compounds | SVOC | 250 to 380° C. |

The VOC content of aerosol hairsprays is substantially determined by the non-aqueous solvents and the propellants, so there is an increasing tendency to resort to water as solvent in place of non-aqueous solvents. However, this replacement of organic solvents has associated problems.

Thus, for example, formulations of film-forming polymers known from, the state of the art which satisfy relevant VOC conditions are not clear and are only sprayable, if at all, after further dilution, so they are of only limited suitability for use in (aerosol) hairsprays. Occasionally, polymer films produced from such compositions do not have the mechanical quality necessary for the desired cosmetic effect on the hair, i.e. they have e.g. an insufficient strengthening action and hold. Such films can also be undesirably sticky.

### STATE OF THE ART

Copolymers based on (meth)acrylate which are water-soluble under alkaline conditions are frequently used in the field of cosmetics as polymers for the hair, especially e.g. as strengthening polymers.

EP-A 331 994 describes hair strengthening compositions containing copolymers of a) 40-60% by weight of C₃-C₁₂-alkyl methacrylates, b) 20-40% by weight of C₄-C₁₀—N-alkyl-substituted acrylamides and c) 10-25% by weight of (meth)acrylic acid. It is preferable to use copolymers of a) isobutyl methacrylate, b) N-tert-octylacrylamide and c) acrylic acid.

DE 2 817 369 describes copolymers in which at least three of the monomer units have a methacrylic acid structure, the copolymers consisting of 22 to 64 mol % of N,N-dimethylaminoethyl methacrylate, 13 to 72 mol % of methyl methacrylate, 6 to 23: mol % of methacrylic acid and 0 to 22 mol % of at least one N-substituted alkyl(meth)acrylamide.

EP-A 62 002 describes terpolymers prepared by the copolymerization of a) 40 to 60% by weight of an N-alkylacrylamide or N-alkylmethacrylamide having 1 to 4 carbon atoms in the alkyl moiety, with b) 35 to 50% by weight of an acrylic acid or methacrylic acid C₁-C₄-hydroxyalkyl ester or, preferably, C₁-C₄-alkyl ester and c) 3 to 11% by weight of an α,β-unsaturated monocarboxylic acid or dicarboxylic acid.

DE 32 27 334 describes copolymers obtained by the free radical copolymerization of 20 to 75 parts by weight of at least one (meth)acrylic acid C₂-C₂₀-alkyl ester, 5 to 50 parts by weight of at least one water-soluble nitrogen-containing monomer having a neutral reaction, 1 to 25 parts by weight of at least one monomer containing cationic groups, and 1 to 25 parts by weight of at least one olefinically unsaturated C₃-C₆-carboxylic acid copolymerizable with a), b) and c), which have a Fikentscher K value of 15 to 75, measured in ethanol at 25-C.

DE 42 23 006 describes hair treating products containing, as film-forming agents, copolymers which are obtainable by the copolymerization of (a) 30 to 80% by weight of an acrylic or methacrylic acid ester, each of which as a homopolymer has a glass transition temperature above 20° C., or of mixtures of acrylic and methacrylic acid esters which, on copolymerization, give copolymers with a glass transition temperature above 20° C., (b) 5 to 25% by weight of acrylic acid, methacrylic acid or mixtures thereof and (c) 10 to 45% by weight of N-vinylpyrrolidone, N-vinyl-caprolactam or mixtures thereof, in the presence of free radical-forming polymerization initiators, and which, in the form of free carboxylic acid groups, have K values (determined according to H. Fikentscher in 1% by weight solution in ethanol at 25° C.) of 10 to 80, wherein the copolymers are prepared by the precipitation polymerization method.

EP-A 805 169 describes copolymers obtainable by the free radical polymerization of a mixture of a) 30 to 72% by weight of t-butyl acrylate or t-butyl methacrylate or a mixture thereof, b) 10 to 28% by weight of acrylic acid or methacrylic acid or a mixture thereof and c) 0 to 60% by weight of at least one other monomer copolymerizable by free radical polymerization.

The unpublished German patent application of file reference 10357486.7 describes copolymers obtainable by the free radical polymerization of a monomer mixture containing

a) tert-butyl acrylate and/or tert-butyl methacrylate,
b) at least one α,β-ethylenically unsaturated compound containing amide groups of the general formula

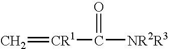

in which
R¹ is H or C₁-C₄-alkyl and
R² and R³ independently of one another are H or C₁-C₄-alkyl, or R² and R³, together with the nitrogen atom to which they are bonded, can also be a 4-membered to 7-membered heterocycle, with the proviso that the sum of the carbon atoms in the radicals R¹, R² and R³ is at most 4, and acrylic acid.

EP-A 256 458 describes copolymers for use as hair fixatives obtained by the free radical polymerization of 20 to 60% by weight of vinylpyrrolidone, 20 to 60% by weight of an acrylamide monoalkylated or dialkylated on the N atom and having 1 to 8 C atoms in the alkyl radical, or mixtures thereof, 5 to 60% by weight of an acrylic or methacrylic acid alkyl or hydroxyalkyl ester having 1 to 4 C atoms in the alkyl radical or 2 to 4 C atoms in the hydroxyalkyl radical, or mixtures of these esters, or 3 to 12% by weight of acrylic acid or methacrylic acid, or 2 to 48% by weight of an acrylic acid or methacrylic acid alkyl or hydroxyalkyl ester having 1 to 4 C atoms in the alkyl radical or 2 to 4 C atoms in the hydroxyalkyl radical, or mixtures of these esters, and 3 to 12% by weight of acrylic acid or methacrylic acid, the percentages by weight being based on the total weight of monomers that is soluble in lower alcohols having 1 to 4 C atoms, said copolymers having a K value of 15 to 75.

WO 95/035087 describes amphoteric hair strengthening polymers consisting of 40-90% by weight of a monomer containing OH groups, 1-20% by weight of a monomer containing acid groups, 1-20% by weight of a monomer containing amino groups and 0-40% by weight of another monomer. The polymer, which is water-insoluble per se, is made water-soluble by various measures such as the addition of neutralizing agents based on acid or amine or by the addition of a low-boiling water-soluble alcohol.

DE 28 17 369 describes copolymers for cosmetics which consist of N,N-dimethyl-aminoethyl methacrylate, methyl methacrylate, methacrylic acid and at least one unsaturated monomer selected from acrylamides or methacrylamides substituted on the nitrogen atom by a linear or branched alkyl radical, monoalkyl ether ethylene (or polyethylene) glycol acrylates or methacrylates, and N-vinylpyrrolidone.

EP-A 0 364 887 describes hair strengthening compositions for aerosols which contain a polymer consisting of 0-60% by weight of C₃-C₁₂-alkyl(meth)acrylate, 15-75% by weight of C₄-C₁₀—N-substituted acrylamide and 20-35% by weight of acrylic acid or methacrylic acid. At least 70% of the carboxyl groups of the polymer are neutralized.

EP-A 0 694 565 describes a process for the homogeneous polymerization of water-insoluble polymers which contain more than 50% by weight of monomers selected from the group comprising C₁-C₁₈-alkyl acrylate or methacrylate, N-substituted acrylamides or methacrylamides and mixtures thereof, in substantially non-aqueous organic solvents, wherein the polymerization initiator used is a water-soluble initiator dissolved in a sufficient amount of water to dissolve it, the amount of water not exceeding 25% by weight of the total solution, and the polymer obtained being characterized by smaller residual monomer contents than those obtained using equivalent amounts of water-insoluble initiators.

U.S. Pat. No. 2,897,172 describes, inter alia, partially hydrolyzed methyl methacrylate/ethyl acrylate copolymers which contain methyl methacrylate, methacrylic acid, acrylic acid and ethyl acrylate units incorporated during the polymerization as a result of hydrolysis.

GB 1410012 describes hair strengthening compositions containing terpolymers of methacrylic acid, methyl methacrylate and methyl acrylate.

### OBJECT AND SOLUTION

One object of the present invention was to provide polymers for cosmetic compositions, especially for the hair, which can easily be formulated as pump or aerosol sprays in solvents or solvent mixtures with an increased water content to give maximum clarity, whose formulations are easily sprayable in the form of small uniform droplets and have a minimum foaming tendency during and after application, and whose subsequently formed films are non-sticky and have good mechanical properties, especially a high stripping resistance.

In addition to good compatibility with the conventional cosmetic ingredients, the polymers applied to the hair should dry rapidly and give the hair a good strength and a prolonged hold, including at high humidity, have a good ease of rinsing and be capable of formulation as optically clear VOC-80 and especially VOC-55 aerosols (i.e. aerosols with a VOC content of at most 80 or 55% by weight, respectively). In particular, the treated hair should have a high luster coupled with a good strength, and good haptic properties, for example a good feel.

The polymers should give the cosmetic compositions for the hair both strengthening properties and conditioning properties.

The above objects have been achieved by the provision of cosmetic compositions containing a copolymer obtainable by the copolymerization of

a) at least one methacrylic acid ester selected from the group comprising methyl methacrylate, ethyl methacrylate, n-propyl methacrylate, i-propyl methacrylate and mixtures thereof,
b) methacrylic acid,
c) acrylic acid,
d) optionally at least one cationic or cationogenic, olefinically unsaturated compound containing amino groups and polymerizable by free radical polymerization,
e) optionally other olefinically unsaturated compounds polymerizable by free radical polymerization,
the copolymerization being carried out in the presence of at least one silicone f) containing polyalkylene oxide if no compound d) is copolymerized,
with the proviso that the copolymerization is carried out in the presence of at least one silicone f) containing polyalkylene oxide if a) is or contains ethyl methacrylate.

Within the framework of the present invention, cationogenic means that compounds referred to in this way carry groups of atoms which can be converted to cationic groups by protonation or especially quaternization.

Within the framework of the present invention, the term “monomer mixture” is used for the total amount of monomers a) to e) to be polymerized by the copolymerization.

The term “monomer mixture” does not mean that the copolymerization is necessarily carried out as a one-pot reaction of the total mixture of all the monomers.

In one preferred embodiment of the invention, a) is selected from methyl methacrylate, ethyl methacrylate and mixtures thereof. Methyl methacrylate is particularly preferred as component a).

The monomer mixture to be polymerized contains preferably at least 50, particularly preferably at least 55 and very particularly preferably at least 60% by weight, and preferably at most 85, particularly preferably at most 82 and very particularly preferably at most 80% by weight of component a).

The monomer mixture to be polymerized contains preferably at least 3, particularly preferably at least 5 and very particularly preferably at least 8% by weight, and preferably at most 30, particularly preferably at most 20 and very particularly preferably at most 15% by weight of methacrylic acid b).

The monomer mixture to be polymerized contains preferably at least 1, particularly preferably at least 3 and very particularly preferably at least 5% by weight, and preferably at most 30, particularly preferably at most 25 and very particularly preferably at most 20% by weight of acrylic acid c).

Within the framework of the present invention, the term alkyl embraces linear and branched alkyl groups. Examples of suitable short-chain alkyl groups are linear or branched C₁-C₁₂-alkyl, preferably C₁-C₆-alkyl and particularly preferably C₁-C₄-alkyl groups. These include especially methyl, ethyl, propyl, isopropyl, n-butyl, 2-butyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-dimethylbutyl, 1,3-dimethyl butyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl, octyl, etc.

Suitable longer-chain C₈-C₃₀-alkyl or C₈-C₃₀-alkenyl groups are linear and branched alkyl or alkenyl groups. Preferably, these are predominantly linear alkyl radicals such as those also occurring in natural or synthetic fatty acids and fatty alcohols, as well as oxo alcohols, which may additionally be mono-, di- or polyunsaturated. These include e.g. n-hexyl(ene), n-heptyl(ene), n-octyl(ene), n-nonyl(ene), n-decyl(ene), n-undecyl(ene), n-dodecyl(ene), n-tridecyl(ene), n-tetradecyl(ene), n-pentadecyl(ene), n-hexadecyl(ene), n-heptadecyl(ene), n-octadecyl(ene), n-nonadecyl(ene), etc. Cycloalkyl is preferably C₅-C₈-cycloalkyl such as cyclopentyl, cyclohexyl, cycloheptyl or cyclooctyl.

Within the framework of the present invention, the term heterocycloalkyl embraces saturated cycloaliphatic groups having in general 4 to 7 and preferably 5 or 6 ring atoms, in which 1 or 2 of the ring carbon atoms have been replaced by heteroatoms selected from the elements oxygen, nitrogen and sulfur, and which can optionally be substituted, it being possible, in the case of substitution, for these heterocycloaliphatic groups to carry 1, 2 or 3, preferably 1 or 2 and particularly preferably 1 substituent selected from alkyl, aryl, COOR, COO⁻M⁺ and NE¹E², alkyl being preferred. Examples of such heterocycloaliphatic groups which may be mentioned are pyrrolidinyl, piperidinyl, 2,2,6,6-tetramethylpiperidinyl, imidazolidinyl, pyrazolidinyl, oxazolidinyl, morpholidinyl, thiazolidinyl, isothiazolidinyl, isoxazolidinyl, piperazinyl, tetrahydro-thiophenyl, tetrahydrofuranyl, tetrahydropyranyl and dioxanyl.

Aryl embraces unsubstituted and substituted aryl groups and is preferably phenyl, tolyl, xylyl, mesityl, naphthyl, fluorenyl, anthracenyl, phenanthrenyl or naphthacenyl, especially phenyl, tolyl, xylyl or mesityl.

Substituted aryl radicals have preferably 1, 2, 3, 4 or 5 and especially 1, 2 or 3 substituents selected from alkyl, alkoxy, carboxyl, carboxylate, trifluoromethyl, —SO₃H, sulfonate, NE¹E², alkylene-N¹E², nitro, cyano and halogen.

Heteroaryl is preferably pyrrolyl, pyrazolyl, imidazolyl, indolyl, carbazolyl, pyridyl, quinolinyl, acridinyl, pyridazinyl, pyrimidinyl or pyrazinyl.

Arylalkyl represents groups containing both alkyl and aryl radicals, said arylalkyl groups being linked either via the aryl radical or via the alkyl radical to the compound carrying them.

In another preferred embodiment of the invention, the compound d) is selected from the group comprising

d1) esters of α,β-ethylenically unsaturated monocarboxylic and dicarboxylic acids with amino alcohols which can be monoalkylated or dialkylated on the amine nitrogen,
d2) amides of α,β-ethylenically unsaturated monocarboxylic and dicarboxylic acids with diamines having at least one primary or secondary amino group,
d3) N,N-diallylamine and derivatives thereof,
d4) vinyl- and allyl-substituted nitrogen heterocycles,
d5) vinyl- and allyl-substituted heteroaromatic compounds and
d6) mixtures thereof.

In one preferred embodiment of the invention, the compounds d) are quaternized to the extent of at least 30, preferably at least 50, particularly preferably at least 70 and especially at least 90 mol % before the polymerization. It is most preferable to quaternize the compounds d) to the extent of at least 99 mol % before they are used for the polymerization with the other compounds.

One preferred embodiment of the invention accordingly consists of cosmetic compositions wherein the compound d) is used for the polymerization in a form quaternized to the extent of at least 50 mol %.

Of course, it is possible to quaternize one part of the compounds d) before the polymerization and another part or all the other parts of the compounds d) after the polymerization.

Methods of quaternizing cationogenic compounds are known to those skilled in the art. Examples of conventional alkylating agents are C₁-C₄-alkyl halides or sulfates, such as ethyl chloride, ethyl bromide, methyl chloride, methyl bromide, dimethyl sulfate and diethyl sulfate. Preferred alkylating agents are methyl chloride and dimethyl and diethyl sulfate, especially methyl chloride.

Suitable compounds d) are d1) the esters of α,β-ethylenically unsaturated monocarboxylic and dicarboxylic acids with amino alcohols. Preferred amino alcohols are C₂-C₁₂-amino alcohols which are C₁-C₈-monoalkylated or dialkylated on the amine nitrogen. Examples of suitable acid components of these esters are acrylic acid, methacrylic acid, fumaric acid, maleic acid, itaconic acid, crotonic acid, maleic anhydride, monobutyl maleate and mixtures thereof. It is preferable to use acrylic acid, methacrylic acid and mixtures thereof. Particularly preferred compounds d) are N-methylaminoethyl(meth)acrylate, N-ethylaminoethyl(meth)acrylate, N-(n-propyl)-aminoethyl (meth)acrylate, N-(n-butyl)aminoethyl(meth)acrylate, N-(tert-butyl)-aminoethyl (meth)acrylate, N,N-dimethylaminomethyl(meth)acrylate, N,N-dimethylaminoethyl (meth)acrylate, N,N-diethylaminoethyl(meth)acrylate, N,N-dimethylaminopropyl (meth)acrylate, N,N-diethylaminopropyl(meth)acrylate and N,N-dimethylaminocyclohexyl (meth)acrylate. N-(tert-butyl)aminoethyl acrylate and N-(tert-butyl)aminoethyl methacrylate are used in particular as compounds d).

Other suitable monomers d) are d2) the amides of the aforementioned α,β-ethylenically unsaturated monocarboxylic and dicarboxylic acids with diamines having at least one primary or secondary-amino group. Diamines having one tertiary and one primary or secondary amino group are preferred. The monomers e) used are preferably N-[2-(dimethylamino)ethyl]acrylamide, N-[2-(dimethylamino)ethyl]-methacrylamide, N-[3-(dimethylamino)propyl]acrylamide, N-[3-(dimethylamino)-propyl]methacrylamide, N-[4-(dimethylamino)butyl]acrylamide, N-[4-(dimethylamino)-butyl]methacrylamide, N-[2-(diethylamino)ethyl]acrylamide, N-[4-(dimethylamino)-cyclohexyl]acrylamide and N-[4-(dimethylamino)cyclohexyl]methacrylamide.

Other suitable monomers d) are d3) N,N-diallylamines, derivatives thereof, e.g. N-alkyl derivatives, and the corresponding acid addition salts. Alkyl is preferably C₁-C₂₄-alkyl here. N,N-diallyl-N-methylamine, for example, is preferred.

Other suitable monomers d) are d4) vinyl- and allyl-substituted nitrogen heterocycles such as N-vinylimidazole, N-vinylimidazole derivatives, e.g. N-vinyl-2-methylimidazole, vinyl- and allyl-substituted heteroaromatic compounds such as 2- and 4-vinylpyridine and 2- and 4-allylpyridine, and salts thereof.

Other suitable monomers d) are N-vinylimidazoles of general formula (II):

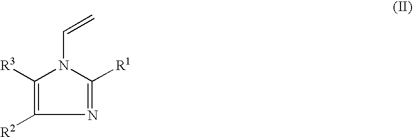

in which R¹ to R³ are hydrogen, C₁-C₄-alkyl or phenyl.

Examples of compounds of general formula (II) can be found in Table 1 below:

TABLE 1

| R¹ | R² | R³ |
|---|---|---|
| H | H | H |
| Me | H | H |
| H | Me | H |
| H | H | Me |
| Me | Me | H |
| H | Me | Me |
| Me | H | Me |
| Ph | H | H |
| H | Ph | H |
| H | H | Ph |
| Ph | Me | H |
| Ph | H | Me |
| Me | Ph | H |
| H | Ph | Me |
| H | Me | Ph |
| Me | H | Ph |

Me = methyl,
Ph = phenyl

Particularly preferably, the compounds of component d) are selected from N-(tert-butylamino)ethyl (meth)acrylate, N,N-dimethylaminoethyl(meth)acrylate, N-[3-(dimethylamino)propyl](meth)acrylamide, N-vinylimidazole and mixtures thereof.

In particular, the compound d) is selected from the group comprising N-[3-(dimethylamino)propyl](meth)acrylamide, N,N-dimethylaminoethyl(meth)acrylate, N-vinylimidazole and mixtures thereof.

It is most preferable to use N-[3-(dimethylamino)propyl]acrylamide and/or N-[3-(dimethylamino)propyl](meth)acrylamide, especially N-[3-(dimethylamino)propyl]-methacrylamide.

In one preferred embodiment of the invention, N-(tert-butylamino)ethyl(meth)acrylate, N,N-dimethylaminoethyl(meth)acrylate, N-[3-(dimethylamino)propyl](meth)acrylamide, N-vinylimidazole and mixtures thereof are used in quaternized form for the polymerization.

If the monomer mixture to be polymerized contains compounds d), it contains preferably at least 0.1, particularly preferably at least 0.5 and very particularly preferably at least 1, and at most 20, preferably at most 10 and particularly preferably at most 5% by weight thereof, based on the total weight of components a) to e).

### Silicones f) Containing Polyalkylene Oxide

If no component d) is copolymerized or if component a) is or contains ethyl methacrylate, the copolymerization is carried out in the presence of at least one silicone f) containing polyalkylene oxide.

However, even if at least one component d) is copolymerized, the copolymerization can be carried out in the presence of silicones f) containing polyalkylene oxide.

Suitable silicones 0 containing polyalkylene oxide are described e.g. in the following publications, whose disclosure is incorporated here to the full extent by way of reference:

DE-PS 16 94 366: This relates to polysiloxane/polyoxyalkylene block copolymers whose polysiloxane block is synthesized in a manner known per se and whose polyoxyalkylene block consists of 25 to 70 percent by weight of a polyoxyalkylene having an average molecular weight of 1600 to 4000 and an ethylene oxide content of 20 to 100 percent by weight, the remainder being propylene oxide and optionally higher alkylene oxides, and 30 to 75 percent by weight of a polyoxyalkylene having an average molecular weight of 400 to 1200 and an ethylene oxide content of 65 to 100 percent by weight, the remainder being propylene oxide and optionally higher alkylene oxides.

DE-OS 25 41 865: The polysiloxane/polyoxyalkylene block copolymers are defined as follows in respect of their polyoxyalkylene blocks: one polyoxyalkylene block has an average molecular weight of 900 to 1300, and 30 to 55% by weight thereof consists of ethylene oxide, the remainder being propylene oxide, and the other polyoxyalkylene block has an average molecular weight of 3800 to 5000, and 30 to 50% by weight thereof consists of ethylene oxide, the remainder being propylene oxide.

EP-A 0 275 563: The block copolymer described comprises three different polyoxyalkylene blocks, namely one block containing 20 to 60% by weight of oxyethylene units and having a molecular weight of 3000 to 5500, another block containing 20 to 60% by weight of oxyethylene units and having a molecular weight of 800 to 2900, and a third block consisting only of polyoxypropylene units and having a molecular weight of 130 to 1200.

Preferred silicones f) containing polyalkylene oxide are described in EP-A 0 670 342. On p. 3, line 22 to p. 4, line 56, EP-A 0 670 342 describes polysiloxanes comprising 1) at least two polyether radicals A and B, the polyoxyalkylene radical A having an average molecular weight of 600 to 5500 and consisting of 20 to 100% by weight of oxyethylene units and 80 to 0% by weight of oxypropylene units, and the polyoxyalkylene radical B having an average molecular weight of 700 to 5000 and consisting of 0 to <20% by weight of oxyethylene units and 100 to 80% by weight of oxypropylene units, and 2) Si-bonded hydrocarbon radicals having 6 to 30 carbon atoms.

Particularly suitable silicone derivatives f) are the compounds known by the INCI name Dimethicone Copolyols or silicone surfactants, e.g. those obtainable under the trade marks Abil® (Goldschmidt), Alkasil® (Rhône-Poulenc), Silicone Polyol Copolymer® (Genesee), Belsil® (Wacker), Silwet® (Witco) or Dow Corning® (Dow Corning). These comprise compounds with the CAS numbers 64365-23-7, 68937-54-2, 68938-545 and 68937-55-3.

Particularly suitable silicone derivatives f) are the compounds described on p. 10, line 24 to p. 12, line 8 and p. 13, line 3 to line 34 of WO 99/04750.

On p. 24, line 22 to p. 26, line 41, WO 01/013884 describes particularly preferred silicones f) containing polyalkylene oxide.

Reference is made to the full extent to the above-cited publications and references from the state of the art.

One particularly preferred embodiment of the invention is a cosmetic composition containing a copolymer obtainable by the copolymerization of
a) at least one methacrylic acid ester selected from the group comprising methyl methacrylate, ethyl methacrylate, n-propyl methacrylate, i-propyl methacrylate and mixtures thereof,
b) methacrylic acid,
c) acrylic acid,
d) at least one cationic or cationogenic, olefinically unsaturated compound containing amino groups and polymerizable by free radical polymerization, and
e) optionally other olefinically unsaturated compounds polymerizable by free radical polymerization,
   the copolymerization being carried out in the presence of a silicone f) containing polyalkylene oxide.

Another preferred embodiment of the invention is a cosmetic composition as described above wherein the copolymer is obtainable by the copolymerization of
a) 50-85% by weight of methyl methacrylate,
b) 5-30% by weight of methacrylic acid,
c) 5-30% by weight of acrylic acid,
d) 0.1-20% by weight of at least one cationic or cationogenic, olefinically unsaturated compound containing amino groups and polymerizable by free radical polymerization, and
e) 0-10% by weight of other olefinically unsaturated compounds polymerizable by free radical polymerization,
   the amounts of components a) to e) adding up to 100% by weight.

Another preferred embodiment of the invention is a cosmetic composition as described above wherein the copolymer is obtainable by the copolymerization of
a) 50-85% by weight of methyl methacrylate and/or ethyl methacrylate,
b) 5-30% by weight of methacrylic acid,
c) 5-30% by weight of acrylic acid,
d) 0.1-20% by weight of at least one cationic or cationogenic, olefinically unsaturated compound containing amino groups and polymerizable by free radical polymerization, and
e) 0-10% by weight of other olefinically unsaturated compounds polymerizable by free radical polymerization,
   the amounts of components a) to e) adding up to 100% by weight and the copolymerization being carried out in the presence of 0.1-3% by weight, based on the sum of components a) to e), of at least one silicone f) containing polyalkylene oxide.

Another preferred embodiment of the invention is a cosmetic composition as described above wherein the copolymer is obtainable by the copolymerization of
a) 50-80% by weight of methyl methacrylate,
b) 8-20% by weight of methacrylic acid,
c) 5-15% by weight of acrylic acid,
d) 0.5-15% by weight of at least one cationic or cationogenic, olefinically unsaturated compound containing amino groups and polymerizable by free radical polymerization, and
e) 0-10% by weight of other olefinically unsaturated compounds polymerizable by free radical polymerization,
   the amounts of components a) to e) adding up to 100% by weight and the copolymerization being carried out in the presence of at least one silicone f) containing polyalkylene oxide.

Another preferred embodiment of the invention is a cosmetic composition as described above wherein the copolymer is obtainable by the copolymerization of
a) 50-80% by weight of methyl methacrylate and/or ethyl methacrylate,
b) 5-30% by weight of methacrylic acid,
c) 5-30% by weight of acrylic acid,
d) 0% by weight of compound d), and
e) 0-10% by weight of other olefinically unsaturated compounds polymerizable by free radical polymerization,
   the amounts of components a) to e) adding up to 100% by weight and the copolymerization being carried out in the presence of at least one silicone f) containing polyalkylene oxide.

Another preferred embodiment of the invention is a cosmetic composition as described above wherein the copolymer is obtainable by the copolymerization of
a) 55-78% by weight of methyl methacrylate and/or ethyl methacrylate,
b) 5-20% by weight of methacrylic acid,
c) 5-20% by weight of acrylic acid,
d) 1-12% by weight of at least one cationic or cationogenic, olefinically unsaturated compound containing amino groups and polymerizable by free radical polymerization, and
e) 0-10% by weight of other olefinically unsaturated compounds polymerizable by free radical polymerization,
   the amounts of components a) to e) adding up to 100% by weight.

Another preferred embodiment of the invention is a cosmetic composition as described above wherein the copolymer is obtainable by the copolymerization of
a) 55-78% by weight of methyl methacrylate and/or ethyl methacrylate,
b) 5-20% by weight of methacrylic acid,
c) 5-20% by weight of acrylic acid,
d) 1-12% by weight of at least one cationic or cationogenic, olefinically unsaturated compound containing amino groups and polymerizable by free radical polymerization, and
e) 0-10% by weight of other olefinically unsaturated compounds polymerizable by free radical polymerization,
   the amounts of components a) to e) adding up to 100% by weight and the copolymerization being carried out in the presence of 0.1-1% by weight, based on the total amount of compounds a) to e), of at least one silicone f) containing polyalkylene oxide.

Another preferred embodiment of the invention is a cosmetic composition as described above wherein the copolymer is obtainable by the copolymerization of
a) 50-78% by weight of methyl methacrylate and/or ethyl methacrylate,
b) 5-20% by weight of methacrylic acid,
c) 5-20% by weight of acrylic acid,
d) 0-15% by weight of at least one cationic or cationogenic, olefinically unsaturated compound containing amino groups and polymerizable by free radical polymerization, and
e) 0-10% by weight of other olefinically unsaturated compounds polymerizable by free radical polymerization,
   the amounts of components a) to e) adding up to 1.00% by weight and the copolymerization being carried out in the presence of 0.1-1% by weight, based on the total amount of compounds a) to e), of at least one silicone f) containing polyalkylene oxide.

One preferred embodiment of the invention is cosmetic compositions as described above wherein the amount of silicone f) containing polyalkylene oxide is 0.05-5% by weight, preferably 0.1-2% by weight, based on the sum of components a) to e), if the copolymerization of the monomer mixture is carried out in the presence of at least one silicone 0 containing polyalkylene oxide.

In one preferred embodiment of the invention, the compounds d) are used in cationic quaternized form for the copolymerization.

### Component e)

The monomer mixture to be polymerized optionally contains other olefinically unsaturated compounds e) polymerizable by free radical polymerization. The component e) is preferably selected from esters of α,β-ethylenically unsaturated monocarboxylic and dicarboxylic acids with C₁-C₃₀-alkanols and C₁-C₃₀-alkanediols, which differ from a), amides of α,β-ethylenically unsaturated monocarboxylic and dicarboxylic acids with C₂-C₃₀-amino alcohols having a primary or secondary amino group, N-vinylamides of saturated monocarboxylic acids, primary amides of α,β-ethylenically unsaturated monocarboxylic acids and N-alkyl and N,N-dialkyl derivatives thereof, esters of vinyl alcohol and allyl alcohol with C₁-C₃₀-monocarboxylic acids, vinyl ethers, vinylaromatics, vinyl halides, vinylidene halides, C₁-C₈-monoolefins, non-aromatic hydrocarbons having at least two conjugated double bonds, and mixtures thereof.

Compounds e) suitable for the copolymerization are given below.

e1) (meth)acrylic or ethacrylic acid esters differing from component a), e.g. methyl ethacrylate, ethyl ethacrylate, n-propyl ethacrylate, i-propyl ethacrylate, n-butyl ethacrylate, tert-butyl ethacrylate, i-butyl ethacrylate, n-butyl(meth)acrylate, tert-butyl (meth)acrylate, i-butyl(meth)acrylate, sec-butyl(meth)acrylate, 2-pentyl(meth)-acrylate, 3-pentyl(meth)acrylate, isopentyl acrylate, neopentyl acrylate, n-octyl (meth)acrylate, 1,1,3,3-tetramethylbutyl(meth)acrylate, ethylhexyl(meth)acrylate, n-nonyl (meth)acrylate, n-decyl(meth)acrylate, n-undecyl(meth)acrylate, tridecyl (meth)acrylate, myristyl(meth)acrylate, pentadecyl(meth)acrylate, palmityl(meth)-acrylate, heptadecyl(meth)acrylate, nonadecyl(meth)acrylate, arachidyl(meth)-acrylate, behenyl(meth)acrylate, lignoceryl(meth)acrylate, cerotyl(meth)acrylate, melissyl(meth)acrylate, palmitoleyl(meth)acrylate, oleyl(meth)acrylate, linolyl (meth)acrylate, linolenyl(meth)acrylate, stearyl(meth)acrylate, lauryl(meth)acrylate, phenoxyethyl acrylate, 4-t-butylcyclohexyl acrylate, cyclohexyl(meth)acrylate, ureido (meth)acrylate, tetrahydrofurfuryl(meth)acrylate and mixtures thereof;
e2) N-vinylamide compounds, e.g. N-vinylformamide, N-vinyl-N-methylformamide, N-vinylacetamide, N-vinyl-N-methylacetamide, N-vinyl-N-ethylacetamide, N-vinyl-propionamide, N-vinyl-N-methylpropionamide and N-vinylbutyramide;
e3) esters of alkanediols with olefinically unsaturated carboxylic acids, e.g. 2-hydroxyethyl acrylate, 2-hydroxyethyl methacrylate, 2-hydroxyethyl ethacrylate, 2-hydroxypropyl acrylate, 2-hydroxypropyl methacrylate, 3-hydroxypropyl acrylate, 3-hydroxypropyl methacrylate, 3-hydroxybutyl acrylate, 3-hydroxybutyl methacrylate, 4-hydroxybutyl acrylate, 4-hydroxybutyl methacrylate, 6-hydroxyhexyl acrylate, 6-hydroxyhexyl methacrylate, 3-hydroxy-2-ethylhexyl acrylate and 3-hydroxy-2-ethylhexyl methacrylate;
e4) amides of amino alcohols with olefinically unsaturated carboxylic acids, e.g. 2-hydroxyethylacrylamide, 2-hydroxyethylmethacrylamide, 2-hydroxyethylethacrylamide, 2-hydroxypropylacrylamide, 2-hydroxypropylmethacrylamide, 3-hydroxypropyl-acrylamide, 3-hydroxypropylmethacrylamide, 3-hydroxybutylacrylamide, 3-hydroxy-butylmethacrylamide, 4-hydroxybutylacrylamide, 4-hydroxybutylmethacrylamide, 6-hydroxyhexylacrylamide, 6-hydroxyhexylmethacrylamide, 3-hydroxy-2-ethylhexyl-acrylamide and 3-hydroxy-2-ethylhexylmethacrylamide;
e5) amides of amines with olefinically unsaturated carboxylic acids, e.g. N-(n-butyl)-methacrylamide, N-(sec-butyl)methacrylamide, N-(tert-butyl)methacrylamide, N-(n-pentyl)(meth)acrylamide, N-(n-hexyl)(meth)acrylamide, N-(n-heptyl)(meth)acrylamide, N-(n-octyl)(meth)acrylamide, N-(tert-octyl)(meth)acrylamide, N-(1,1,3,3-tetramethyl-butyl)(meth)acrylamide, N-ethylhexyl(meth)acrylamide, N-(n-nonyl)(meth)acrylamide, N-(n-decyl)(meth)acrylamide, N-(n-undecyl)(meth)acrylamide, N-tridecyl(meth)-acrylamide, N-myristyl(meth)acrylamide, N-pentadecyl(meth)acrylamide, N-palmityl-(meth)acrylamide, N-heptadecyl(meth)acrylamide, N-nonadecyl(meth)acrylamide, N-arachidyl(meth)acrylamide, N-behenyl(meth)acrylamide, N-lignoceryl(meth)acrylamide, N-cerotyl(meth)acrylamide, N-melissyl(meth)acrylamide, N-palmitoleyl(meth)-acrylamide, N-oleyl(meth)acrylamide, N-linolyl(meth)acrylamide, N-linolenyl(meth)-acrylamide, N-stearyl(meth)acrylamide and N-lauryl(meth)acrylamide;
e6) vinyl esters, e.g. vinyl acetate, vinyl propionate, vinyl butyrate and mixtures thereof;
e7) ethylene, propylene, isobutylene, butadiene, styrene, α-methylstyrene, acrylonitrile, methacrylonitrile, vinyl chloride, vinylidene chloride, vinyl fluoride and vinylidene fluoride;
e8) compounds having per molecule at least 2 non-conjugated double bonds polymerizable by free radical polymerization, which can occasionally also be referred to as crosslinking agents.

Examples, of suitable components e8) are acrylic acid esters, methacrylic acid esters, allyl ethers or vinyl ethers of at least dihydric alcohols. The OH groups of the alcohols on which they are based can be wholly or partially etherified or esterified, but the components e8) contain at least two unsaturated groups polymerizable by free radical polymerization. Examples of the alcohols-on which they are based are dihydric alcohols such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentyl glycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, hydroxypivalic acid neopentyl glycol monoester, 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiopentane-1,5-diol, and polyethylene glycols, polypropylene glycols and polytetrahydrofurans each having molecular weights of 200 to 10,000.

As well as ethylene oxide or propylene oxide homopolymers, it is also possible to use block copolymers of ethylene oxide or propylene oxide, or copolymers containing incorporated ethylene oxide and propylene oxide groups.

Examples of alcohols on which the components e8) are based and which have more than two OH groups are trimethylolpropane, glycerol, pentaerythritol, 1,2,5-pentanetriol, 1,2,6-hexanetriol, triethoxycyanuric acid, sorbitan, and sugars such as sucrose, glucose and mannose. Other preferred polyhydric alcohols in this context are disaccharides and trisaccharides.

Of course, the polyhydric alcohols can also be used after reaction with ethylene oxide or propylene oxide as the corresponding ethoxylates or propoxylates. The polyhydric alcohols can also be converted first to the corresponding glycidyl ethers by reaction with epichlorohydrin.

Other suitable components e8) are esters of vinyl alcohol or monohydric unsaturated alcohols with olefinically unsaturated C₃- to C₆-carboxylic acids, e.g. acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-octadecen-1-ol. It is also possible, however, to esterify the monohydric unsaturated alcohols with polybasic carboxylic acids, e.g. malonic acid, tartaric acid, trimellitic acid, phthalic acid, terephthalic acid, citric acid or succinic acid.

Other suitable components e8) are esters of unsaturated carboxylic acids, e.g. oleic acid, crotonic acid, cinnamic acid or 10-undecenoic acid, with the polyhydric alcohols described above.

Other suitable components e8) are linear, branched or cyclic aliphatic or aromatic hydrocarbons having at least two double bonds which, in the case of aliphatic hydrocarbons, must not be conjugated, e.g. divinylbenzene, divinyltoluene, 1,7-octadiene, 1,9-decadiene, 4-vinyl-1-cyclohexene, trivinylcyclohexane, or polybutadienes having molecular weights of 200 to 20,000.

Other suitable components e8) are the amides of (meth)acrylic acid, itaconic acid and maleic acid with N-allylamines of at least dibasic amines. Examples of such amines are 1,2-diaminomethane, 1,2-diaminoethane, 1,3-diaminopropane, 1,4-diamino-butane, 1,6-diaminohexane, 1,12-dodecanediamine, piperazine, diethylenetriamine or isophoronediamine. Other suitable compounds are the amides of allylamine with unsaturated carboxylic acids such as acrylic acid, methacrylic acid, itaconic acid, maleic acid or at least dibasic carboxylic acids, as described above.

Triallylamine and triallylmonoalkylammonium salts, e.g. triallylmethylammonium chloride or methylsulfate, are also suitable as components e8).

Also suitable are N-vinyl compounds of urea derivatives, at least dibasic amides, cyanurates or urethanes, e.g. those of urea, ethyleneurea, propyleneurea or tartaric acid diamide, examples being N,N′-divinylethyleneurea or N,N′-divinylpropyleneurea.

Alkylenebisacrylamides such as methylenebisacrylamide, and N,N′-(2,2)butane and 1,1′-bis(3,3′-vinylbenzimidazolin-2-one)-1,4-butane are also suitable.

Examples of other suitable components e8) are alkylene glycol di(meth)acrylates such as ethylene glycol diacrylate, ethylene glycol dimethacrylate, tetraethylene glycol acrylate, tetraethylene glycol dimethacrylate, diethylene glycol acrylate, diethylene glycol methacrylate, vinyl acrylate, allyl acrylate, allyl methacrylate, divinyldioxane, pentaerythrityl allyl ether and mixtures of these components.

Other suitable components e8) are tetraallylsilane or tetravinylsilane.

Other suitable components e8) are polyurethaneacrylates, which can optionally also contain silicone groups. Polyurethaneacrylates and their various preparative methods are known to those skilled in the art.

Examples of components e8) that are particularly preferably used are methylene-bisacrylamide, triallylamine and triallylalkylammonium salts, divinylimidazole, pentaerythrityl triallyl ether, N,N′-divinylethyleneurea, reaction products of polyhydric alcohols with acrylic acid or methacrylic acid, methacrylic-acid esters and acrylic acid esters of polyalkylene oxides or of polyhydric alcohols which have been reacted with ethylene oxide and/or propylene oxide and/or epichlorohydrin, and especially diallyl ether or di(meth)acrylic acid esters of polyethylene glycol.

Very particularly preferred components e8) are pentaerythrityl triallyl ether, methylene-bisacrylamide, N,N′-divinylethyleneurea, triallylamine and triallylmonoalkylammonium salts, and acrylic acid esters of glycol, butanediol, trimethylolpropane or glycerol, or acrylic acid esters of glycol, butanediol, trimethylolpropane or glycerol which have been reacted with ethylene oxide and/or epichlorohydrin.

Of course, it is also possible to use mixtures of the aforesaid compounds. The component e8) is preferably soluble in the reaction medium. If the solubility of the component e8) in the reaction medium is low, it can be dissolved in a monomer or a monomer mixture, orbit can be metered in as a solution in a solvent that is miscible with the reaction medium. Particularly preferred components e8) are those which are soluble in the monomer mixture.

If the component e8) is used to prepare the polymer A according to the invention, the amount used is at least 0.01, preferably at least 0.05 and particularly preferably at least 0.1, and at most 5; preferably at most 2 and particularly preferably at most 1% by weight, based on the total amount of components a) to e).

e9) unsubstituted N-vinyllactams and N-vinyllactam derivatives which can have e.g. one or more C₁-C₆-alkyl substituents such as methyl, ethyl, n-propyl, isopropyl, n-butyl, sec-butyl, tert-butyl, etc., examples being N-vinylpyrrolidone, N-vinylpiperidone, N-vinylcaprolactam, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam, N-vinyl-7-ethyl-2-caprolactam and mixtures thereof.

In one preferred embodiment of the invention, however, the monomer mixtures to be polymerized contain at most 5, preferably at most 3, particularly preferably at most 1 and very particularly preferably at most 0.1% by weight of N-vinyllactams. In one preferred embodiment of the invention, no N-vinyllactams are copolymerized.

The additional monomers e) mentioned above can be used individually or in the form of any desired mixtures. Based on the total weight of components a) to e), the monomer mixture to be polymerized contains at most 10% by weight, particularly preferably at most 7% by weight and very particularly preferably at most 5% by weight of monomer e) incorporated during the polymerization.

### Preparation of the Copolymers According to the Invention

The copolymers according to the invention can be prepared e.g. by solution, precipitation, suspension or emulsion polymerization. Such methods are known in principle to those skilled in the art. Preparation by solution polymerization is preferred. It is preferable to prepare the copolymers by free radical solution polymerization.

Preferred solvents for the copolymerization are alcoholic or alcoholic-aqueous solvents, such as ethanol and mixtures of ethanol with water, isopropanol and mixtures of isopropanol with water and/or other alcohols such as methanol, n-propanol, n-butanol, sec-butanol, tert-butanol, n-hexanol and cyclohexanol, and with glycols such as ethylene glycol, propylene glycol and butylene glycol and the methyl or ethyl ethers of dihydric alcohols such as diethylene glycol, triethylene glycol, polyethylene glycols having number-average molecular weights of up to about 3000, glycerol and dioxane.

Polymerization in an alcohol, e.g. ethanol, in an ethanol/water mixture, in isopropanol or in an isopropanol/water mixture is particularly preferred.

The polymerization temperatures preferably range from about 30 to 140° C., particularly preferably from 40 to 120° C. and very particularly preferably from 65 to 120° C. The polymerization conventionally takes place under atmospheric pressure, but it can also proceed under reduced or elevated pressure. A suitable pressure range is between 1 and 5 bar.

The polymerization is preferably carried out under an inert gas atmosphere.

The monomers can be copolymerized with the aid of free radical-forming initiators. The initiators used for the free radical polymerization can be the peroxo and/or azo compounds conventionally used for this purpose, e.g. alkali metal or ammonium peroxydisulfates, diacetyl peroxide, dibenzoyl peroxide, succinyl peroxide, ditert-butyl peroxide, tert-butyl perbenzoate, tert-butyl perpivalate, tert-butyl peroxy-2-ethyl-hexanoate, tert-butyl permaleate, cumene hydroperoxide, diisopropyl peroxy-dicarbamate, bis(o-toluoyl)peroxide, didecanoyl peroxide, dioctanoyl peroxide, dilauroyl peroxide, tert-butyl perisobutyrate, tert-butyl peracetate, ditert-amyl peroxide, tert-butyl hydroperoxide, azobisisobutyronitrile, 2,2′-azobis(2-amidinopropane) hydrochloride (Wako V-50@), 2,2′-azobis[2-(2-imidazolin-2-yl)propane] (Wako VA-061®), 2,2′-azobis(2-methylbutyronitrile) (Wako V-59®), dimethyl 2,2′-azobis(2-methylpropionate) (Wako V-601®), 2,2′-azobis(2,4-dimethylvaleronitrile), 1,1′-azobis(1-cyclohexanecarbonitrile), 4,4′-azobis(4-cyanovaleric acid) or 2-(carbamoylazo)-isobutyronitrile.

Initiator mixtures or redox initiator systems are also suitable, examples being ascorbic acid/iron(II) sulfate/sodium peroxodisulfate, tert-butyl hydroperoxide/sodium disulfite, tert-butyl hydroperoxide/sodium hydroxymethanesulfinate, H₂O₂/Cu and H₂O₂/ascorbic acid.

Suitable oxidizing agents for redox initiator systems are essentially the peroxides mentioned above. Corresponding reducing agents which can be used are sulfur compounds in a low oxidation state, such as alkali metal sulfites, e.g. potassium and/or sodium sulfite, alkali metal hydrogen sulfites, e.g. potassium and/or sodium hydrogen sulfite, alkali metal metabisulfites, e.g. potassium and/or sodium metabisulfite, formaldehydesulfoxylates, e.g. potassium and/or sodium formaldehyde-sulfoxylate, alkali metal salts, especially potassium and/or sodium salts, of aliphatic sulfinic acids, alkali metal hydrogen sulfides, e.g. potassium and/or sodium hydrogen sulfide, salts of polyvalent metals, such as iron(II) sulfate, iron(II) ammonium sulfate and iron(II) phosphate, enediols such as dihydroxymaleic acid, benzoin and/or ascorbic acid, reducing saccharides such as sorbose, glucose and fructose, and/or dihydroxyacetone.

It can also be advantageous to use mixtures of Water-soluble and sparingly water-soluble or water-insoluble initiators.

Suitable initiators are described in chapters 20 and 21 of Macromolecules, vol. 2, 2nd ed., H. G. Elias, Plenum Press, 1984, New York, which is incorporated here to the full extent by way of reference. Furthermore, suitable photoinitiators are described in S. P. Pappas, J. Rad. Cur., July 1987, p. 6, which is incorporated here to the full extent by way of reference.

The initiators are conventionally used in amounts of 0.01 to 10 and preferably of 0.02 to 5% by weight, based on the total amount of monomers to be polymerized.

The K value of the polymers ranges from 15 to 120, preferably from 25 to 75, particularly preferably from 25 to 55 and very particularly preferably from 30 to 50 (as determined according to Fikentscher, Cellulosechemie (Cellulose Chemistry), vol. 13, pp 58 to 64 (1932)). Those skilled in the art are familiar with possible ways of adjusting the K value of polymers to within a desired range, examples being the polymerization temperature, the amount of initiator and/or the use of chain transfer reagents.

The molecular weight can be adjusted by carrying out the polymerization in the presence of at least one chain transfer reagent (regulator). Chain transfer reagents which can be used are the conventional compounds known to those skilled in the art, such as sulfur compounds, e.g. mercaptoethanol, 2-ethylhexyl thioglycolate, thio-glycolic acid, alkanethiols, cysteine and acetylcysteine, and tribromochloromethane or other compounds which have a regulating effect on the molecular weight of the polymers obtained.

The regulators are conventionally used in amounts of 0.1 to 5% by weight, especially of 0.25 to 2% by weight, based on the monomers to be polymerized. Conventionally, the regulators are added to the polymerization together with the monomers.

To achieve the purest possible copolymers with a low residual monomer content, the polymerization (main polymerization) can be followed by at least one post-polymerization step. The postpolymerization can take place in the presence of the same initiator system as the main polymerization or a different initiator system. Preferably, the postpolymerization takes place at the same temperature as the main polymerization or, preferably, at a higher temperature. If desired, the reaction mixture can be subjected to steam stripping or steam distillation after the polymerization or between the first and second polymerization steps.

The copolymerization is carried out according to the conventional process techniques of solution polymerization, e.g. by so-called batch polymerization, Wherein the monomers and optionally polymerization regulators and initiators are placed in a solvent and heated to the polymerization temperature. The reaction mixture is preferably stirred at the polymerization temperature until the monomer conversion is more than 99.8% by weight. In these processes, it is also optionally possible to add the initiators only after the polymerization temperature has been reached.

Other process variants are fed batch methods, which are used in preference. Here, all or part of the individual reactants or of all the reactants is added to a reaction mixture batchwise or continuously and together or in separate feed streams. Thus, for example, a solution of the polymerization regulator and an initiator solution can optionally be added continuously or batchwise to a mixture of the monomers and a solvent at the polymerization temperature over a given period of time. It is also possible, however, to meter a mixture of initiator and optionally regulator into the initial ingredients heated to the polymerization temperature. Another variant consists in adding the initiator to the initial ingredients below or at the polymerization temperature and, if a regulator is to be used, to introduce the regulator or a solution of the regulator into the reaction mixture, over a predetermined period of time, only after the polymerization temperature has been reached.

The organic solvent used in the preparation of the polymers can be removed by conventional methods known to those skilled in the art, e.g. distillation under reduced pressure. After the polymerization process, the mixtures formed in the polymerization can be subjected to a physical or chemical aftertreatment. Examples of such processes are the known residual monomer reduction processes, e.g. aftertreatment by the addition of polymerization initiators or mixtures of several polymerization initiators at appropriate temperatures or by heating of the polymerization solution to temperatures above the polymerization temperature, aftertreatment of the polymer solution with steam or by stripping with nitrogen or by treatment of the reaction mixture with oxidizing or reducing reagents, adsorption processes such as the adsorption of impurities on selected media, e.g. active charcoal, or ultrafiltration. If N-vinyllactams, especially N-vinylpyrrolidone, are copolymerized as component e), it is advantageous to remove monomeric vinyllactam from the resulting copolymers by steam distillation.

The process can also be followed by the known work-up steps, e.g. suitable drying processes such as spray drying, freeze drying or drum drying, or agglomeration processes following drying. The mixtures low in residual monomers which are obtained by the process according to the invention can also be directly marketed.

### Neutralization

The copolymers according to the invention can be partially or completely neutralized. Partial neutralization is advantageous particularly if the copolymers are used in cosmetic compositions for the hair. The term “degree of neutralization” is known to those skilled in the art. It indicates how many of the neutralizable groups have been neutralized and ranges from 0 to 1. The degree of neutralization is conventionally given in %.

In preferred embodiments the polymers are neutralized e.g. to the extent of at least 30, preferably at least 35, more preferably at least 40, particularly preferably at least 45, very particularly preferably at least 50 and especially at least 55%.

In one very particularly preferred embodiment the polymers are neutralized to the extent of at least 60%. It is most preferable to neutralize the copolymers to an extent ranging from 60 to 90%.

The neutralization can take place during or after the polymerization.

The neutralization can be effected e.g. with
a mono-, di- or trialkanolamine having 2 to 5 carbon atoms in the alkanol radical, which is optionally present in etherified form, e.g. mono-, di- and triethanolamine, mono-, di- and tri-n-propanolamine, mono-, di- and triisopropanolamine, 2-amino-2-methylpropanol (AMP) and di(2-methoxyethyl)amine,
an alkanediolamine having 2 to 5 carbon atoms, e.g. 2-amino-2-methylpropane-1,3-diol and 2-amino-2-ethylpropane-1,3-diol, or
a primary, secondary or tertiary alkylamine having a total of 5 to 10 carbon atoms, e.g. N,N-diethylpropylamine or 3-diethylamino-1-propylamine.

Alkali metal hydroxides suitable for the neutralization are particularly sodium or potassium hydroxide; ammonium hydroxide is also suitable.

Good neutralization results are frequently obtained with 2-amino-2-methylpropanol (AMP), N-methyldiethanolamine, triethanolamine, triisopropanolamine, 2-amino-2-ethylpropane-1,3-diol, N,N-dimethylaminoethanol or 3-diethylamino-1-propylamine.

In one preferred embodiment of the invention, amines containing hydroxyl groups, from the group comprising N,N-dimethylethanolamine, N-methyldiethanolamine, triethanolamine, 2-amino-2-methylpropanol (AMP) and mixtures thereof, are selected for the neutralization.

Alkanolamines carrying primary or tertiary amino groups can exhibit advantageous effects here.

Silicone polymers containing amino groups are also suitable for neutralizing the copolymers in the compositions according to the invention. Examples of suitable silicone polymers containing amino groups are the silicone/aminopolyalkylene oxide block copolymers of WO 97/32917 and the products Silsoft® A-843 (Dimethicone Bisamino Hydroxypropyl Copolyol) and Silsoft®A-858 (Trimethylsilyl Amodimethicone Copolymer) (both from Witco). The neutralizing polymers of EP-A 1 035 144, and especially the silicone-containing neutralizing polymers as claimed in claim 12 of EP-A 1 035 144, are also suitable.

The invention also provides a process for the preparation of a copolymer according to, the invention wherein the copolymerization is carried out as a solution polymerization
in a solvent comprising a mixture of at most 99% by weight of alcohol and at least 1% by weight of water,
at 65-120° C.,
in the presence of at least one organic initiator,
at least 30 mol % of the acid groups of the copolymer being neutralized with at least one amine containing hydroxyl groups when the residual monomer content is at most 5, preferably at most 1 and particularly preferably at most 0.2% by weight, based on the polymerization solution, and
at least 90% of the organic solvent then being removed by steam distillation when the residual monomer content is at most 0.2% by weight, based on the polymerization solution.

One preferred embodiment of the invention is a process described as above wherein at least 95% of the organic solvent is removed by steam distillation.

One preferred embodiment of the invention is a process described as above wherein 10 to 40% by weight of ethanol is added to the product obtained after the steam distillation, based on the resulting product.

### Quaternization

If the copolymers contain quaternizable groups that are not yet quaternized, e.g. tertiary amino groups, it can be advantageous to quaternize them. The quaternization is known to those skilled in the art and takes place in conventional manner after the polymerization, e.g. when the conversion is at least 90, preferably at least 95, particularly preferably at least 99 and very particularly preferably at least 99.8% by weight. Conventional and preferred quaternizing agents have already been mentioned previously.

### Cosmetic Compositions

The copolymers described above are outstandingly suitable for the preparation of cosmetic compositions; especially for the skin and/or hair, where they serve e.g. as polymeric film-forming agents. They can be universally used and formulated in a wide variety of cosmetic compositions, preferably for the skin and/or hair, and are compatible with the conventional components.

In the field of hair cosmetics the polymers are suitable e.g. for producing elastic hair styles coupled with a good strengthening action, including at high humidity. The copolymers according to the invention are distinguished by good propellant gas compatibility, by good solubility in aqueous-alcoholic solvent mixtures, in particular by their suitability for use as optically clear, low-VOC formulations; and by good ease of rinsing and ease of combing without a flaking effect. In addition, they improve hair treated therewith in terms of its sensory properties such as feel, volume, manageability, etc. Hairspray formulations, based on the copolymers according to the invention are distinguished by good sprayability and good rheological properties, extremely low stickiness and high stripping resistance of the resulting films. The cosmetic compositions, preferably for the skin and/or hair, containing the copolymers according to the invention do not tend to foam after application. Apart from good compatibility with the conventional cosmetic ingredients, the copolymers dry rapidly after application.

### Cosmetically Acceptable Carrier B)

The cosmetic compositions according to the invention are preferably aqueous compositions containing at least 10, preferably at least 20 and particularly preferably at least 30% by weight of Water. In addition to water and the polymers A, the cosmetic compositions according to the invention also contain at least one cosmetically acceptable carrier B) selected from
i) water-miscible organic solvents, preferably C₂-C₄-alkanols and especially ethanol,
ii) oils, fats and waxes,
iii) esters of C₆-C₃₀-monocarboxylic acids with mono-, di- or trihydric alcohols, which differ from ii),
iv) saturated acyclic and cyclic hydrocarbons,
v) fatty acids,
vi) fatty alcohols,
vii) propellants (propellant gases) and
viii) mixtures thereof.

Suitable carriers B and other active substances and additives that can advantageously be used are described in detail below.

Suitable cosmetically and pharmaceutically acceptable oil or fat components B) are described in Karl-Heinz Schrader, Grundlagen und Rezepturen der Kosmetika (Cosmetic Principles and Formulations), 2nd edition, Verlag Hüthig, Heidelberg, pp 319-355, which is incorporated here by way of reference.

The compositions according to the invention can contain e.g. an oil or fat component B) selected from

hydrocarbons of low polarity, such as mineral oils; linear saturated hydrocarbons preferably having more than 8 C atoms, such as tetradecane, hexadecane, octadecane, etc.; cyclic hydrocarbons such as decahydronaphthalene; branched hydrocarbons; animal and vegetable oils; waxes; wax esters; petrolatum; esters, preferably fatty acid esters, e.g. the esters of C₁-C₂₄-monoalcohols with C₁-C₂₂-monocarboxylic acids, such as isopropyl isostearate, n-propyl myristate, isopropyl myristate, n-propyl palmitate, isopropyl palmitate, hexacosanyl palmitate, octacosanyl palmitate, triacontanyl palmitate, ditriacontanyl palmitate, tetratriacontanyl palmitate, hexacosanyl stearate, octacosanyl stearate, triacontanyl stearate, ditriacontanyl stearate and tetratriacontanyl stearate; salicylates such as C₁-C₁₀-salicylates, e.g. octyl salicylate; benzoate esters such as C₁₀-C₁₅-alkyl benzoates and benzyl benzoate; other cosmetic esters such as fatty acid triglycerides, propylene glycol monolaurate, polyethylene glycol monolaurate, C₁₀-C₁₅-alkyl lactates, etc.; and mixtures thereof. Preferred oil or fat components, B) are selected from paraffin and paraffin oils; petrolatum; natural fats and oils such as castor oil, soya oil, groundnut oil, olive oil, sunflower oil, sesame oil, avocado oil, cacao butter, almond oil, peach kernel oil, castor oil, cod-liver oil, lard, spermaceti, spermaceti oil, sperm oil, wheatgerm oil, macadamia nut oil, evening primrose oil and jojoba oil; fatty alcohols such as lauryl alcohol, myristyl alcohol, cetyl alcohol, stearyl alcohol and oleyl alcohol; fatty acids such as myristic acid, stearic acid, palmitic acid, oleic acid, linoleic acid, linolenic acid and saturated, unsaturated and substituted fatty acids that differ from these; waxes such as beeswax, carnauba wax, candelilla wax and spermaceti; and mixtures of the aforementioned oil or fat components.

Suitable silicone oils B) are e.g. linear polydimethylsiloxanes, poly(methylphenyl-siloxanes), cyclic siloxanes and mixtures thereof. The number-average molecular weight of the polydimethylsiloxanes and poly(methylphenylsiloxanes) preferably ranges from about 1000 to 150,000 g/mol. Preferred cyclic siloxanes have 4- to 8-membered rings. Suitable cyclic siloxanes are commercially available e.g. under the name Cyclomethicone.

Suitable hydrophilic carriers B) are selected from water and mono-, di- or polyhydric alcohols preferably having 1 to 8 carbon atoms, such as ethanol, n-propanol, isopropanol, propylene glycol, glycerol, sorbitol, etc.

The cosmetic compositions according to the invention can be cosmetic products for the skin or hair or dermatological, hygiene or pharmaceutical products. On the basis of their film-forming and flexible properties, the copolymers described above are particularly suitable as additives for skin and hair cosmetics.

The compositions according to the invention which contain the copolymers preferably take the form of a spray, gel, foam, ointment, cream, emulsion, suspension, lotion, milk or paste. If desired, liposomes or microspheres can also be used. In particular, the cosmetic compositions according to the invention take the form of aerosol sprays or mousses.

The compositions according to the invention preferably contain at least one copolymer defined as above, at least one carrier B defined as above and at least one constituent differing from these which is preferably selected from cosmetically active substances, emulsifiers, surfactants, preservatives, perfume oils, thickeners, hair polymers, hair and skin conditioners, graft polymers, water-soluble or dispersible silicone-containing polymers, sunscreen agents, bleaching agents, gelling agents, nurturing agents, colorants, toners, browning ingredients, dyestuffs, pigments, texturing agents, humectants, superfatting agents, collagen, protein hydrolyzates, lipids, antioxidants, defoamers, antistatics, emollients and plasticizers.

The compositions according to the invention preferably have a pH of 2.0 to 9.3. The pH range is particularly preferably between 4 and 8.5. Additional co-solvents which can be present are organic solvents or mixtures of solvents with a boiling point below 400° C., in an amount of 0.1 to 15% by weight and preferably of 1 to 10% by weight. Particularly suitable additional co-solvents are unbranched or branched hydrocarbons such as pentane, hexane and isopentane, and cyclic hydrocarbons such as cyclopentane and cyclohexane. Other particularly preferred water-soluble solvents are glycerol, ethylene glycol and propylene glycol, in an amount of up to 30% by weight.

In one preferred embodiment of the invention the compositions according to the invention contain at most 80% by weight, preferably at most 55% by weight and particularly preferably at most 35% by weight of volatile organic components. Thus one preferred embodiment of the invention is cosmetic compositions, preferably for the hair, which comply with the low-VOC standard, i.e. the VOC-80 and, preferably, VOC-55 standard.

The copolymers are preferably used especially in hairspray compositions containing the following constituents:
partially or completely neutralized copolymer according to the invention;
water;
cosmetically conventional organic solvent, e.g. ethanol, isopropanol or dimethoxymethane, or alternatively acetone, n-propanol, n-butanol, 2-methoxy-propan-1-ol, n-pentane, n-hexane, cyclohexane, n-heptane, n-octane or dichloromethane, or mixtures thereof;
cosmetically conventional propellant, e.g. n-propane, isopropane, n-butane, i-butane, 2,2-dimethylbutane, n-pentane, isopentane, dimethyl ether, difluoro-ethane, fluorotrichloromethane, dichlorodifluoromethane or dichlorotetrafluoro-ethane, HFC-152 A (1,1-difluoroethane), HFC-134a (1,1,2,2-tetrafluoro-ethane), N₂, N₂O or CO₂, or mixtures thereof.

To neutralize the copolymers according to the invention and adjust the pH of the cosmetic compositions, preferably for the skin and/or hair, it is advantageous to use alkanolamines; preferred alkanolamines being those with primary and/or tertiary amino groups. Examples are aminomethylpropanol, N-methyldiethanolamine, N-methyldiisopropanolamine, ethanolamine, N,N-dimethylethanolamine, N-lauryl-diethanolamine, triethanolamine, triisopropanolamine, etc. Preferred alkanolamines have already been mentioned in the preparation of the copolymers according to the invention.

The neutralization can also be effected using alkali metal hydroxides (e.g. NaOH, preferably KOH) and other bases (e.g. histidine, arginine, lysine; ethylenediamines, diethylenetriamine, melamine, benzoguanamine). All the bases indicated can be used on their own or as a mixture with other bases for neutralizing acid-containing cosmetic products.

The present invention accordingly provides aqueous cosmetic compositions, preferably for the hair, which contain, apart form the at least one copolymer and the carrier B, at least one other active substance or additive selected from the group comprising viscosity modifiers, hair nurturing substances, hair strengtheners, silicone compounds, sunscreen agents, fats, oils, waxes, preservatives, pigments, soluble dyestuffs, particulate substances and surfactants.

In one preferred embodiment, cosmetic compositions for the hair according to the invention contain
i) 0.05 to 20% by weight of at least one copolymer according to the invention,
ii) 20 to 99.95% by weight of water and/or alcohol,
iii) 0 to 50% by weight of at least one propellant gas,
iv) 0 to 50% by weight of at least one emulsifier,
v) 0 to 3% by weight of at least one-thickener, and
vi) up to 25% by weight of other constituents.

Alcohol is understood as meaning any of the aforementioned alcohols conventionally used in cosmetics, e.g. ethanol, isopropanol or n-propanol.

### Propellants (Propellant Gases)

The compounds used in particular among those mentioned as propellants (propellant gases) are the hydrocarbons, especially propane, n-butane, n-pentane and mixtures thereof, as well as dimethyl ether and difluoroethane. One or more of the chlorinated hydrocarbons mentioned are optionally used concomitantly in propellant mixtures, but only in small amounts of, for instance, up to 20% by weight, based on the propellant mixture.

The cosmetic compositions according to the invention, preferably for the hair, are also particularly suitable as pump spray compositions without the addition of propellants, or else as aerosol sprays with conventional compressed gases, such as nitrogen, compressed air or carbon dioxide, as propellant.

A water-containing standard aerosol spray formulation comprises e.g. the following constituents:
polymer according to the invention, 60 to 90% neutralized with AMP,
alcohol,
water and
dimethyl ether and/or propane/n-butane and/or propane/isobutane.

The total amount of volatile organic components here is preferably at most, 80 and particularly preferably at most 55% by weight of the composition.

The cosmetic compositions according to the invention, preferably for the hair, preferably contain at least one copolymer according to the invention, at least one cosmetically acceptable carrier B) defined as above, and at least one other active substance or additive differing from these which is selected from cosmetically active substances, emulsifiers, surfactants, preservatives, perfume oils, thickeners, hair polymers, hair conditioners, graft polymers, water-soluble or dispersible silicone-containing polymers, sunscreen agents, bleaching agents, gelling agents, nurturing agents, colorants, toners, browning ingredients, dyestuffs, pigments, texturizers, humectants, superfatting agents, collagen, protein hydrolyzates, lipids, antioxidants, defoamers, antistatics, emollients, lanolin components, protein hydrolyzates and plasticizers.

### Other Polymers

To specifically adjust the properties of cosmetic compositions, preferably for the hair, it can be advantageous to use the copolymers according to the invention in a mixture with other polymers conventionally used in (hair) cosmetics.

In another preferred embodiment the composition according to the invention contains 0.01 to 156% by weight and preferably 0.5 to 10% by weight of at least one synthetic or natural polymer. Natural polymers are also understood as meaning chemically modified polymers of natural origin. Film-forming polymers are understood as meaning polymers which, when used in 0.01 to 5% aqueous, alcoholic or aqueous-alcoholic solution, are capable of depositing a polymer film on the hair.

Anionic, cationic, amphoteric, zwitterionic and neutral polymers, for example, are suitable as said other conventional polymers.
Examples of said other polymers are
copolymers of ethyl acrylate and methacrylic acid,
copolymers of N-tert-butylacrylamide, ethyl acrylate and acrylic acid,
polyvinylpyrrolidones,
polyvinylcaprolactams,
polyurethanes,
copolymers of acrylic acid, methyl methacrylate, octylacrylamide, butylaminoethyl methacrylate and hydroxypropyl methacrylate,
copolymers of vinyl acetate and crotonic acid and/or (vinyl) neodecanoate,
copolymers of vinyl acetate and/or vinyl propionate and N-vinylpyrrolidone,
carboxy-functional copolymers of vinylpyrrolidone, t-butyl acrylate and methacrylic acid, and
copolymers of tert-butyl acrylate, methacrylic acid and Dimethicone Copolyol.

Surprisingly, it has been found that cosmetic compositions, preferably for the skin and/or hair, which contain the copolymers in combination with other polymers have unexpected properties. Particularly as regards their cosmetic properties taken overall, the cosmetic compositions according to the invention, preferably for the hair, are superior to the compositions of the state of the art.

Copolymers of ethyl acrylate and methacrylic acid (INCI name, Acrylates Copolymer) are obtainable e.g. as the commercial products Luviflex® Soft (BASF).

Copolymers of N-tert-butylacrylamide, ethyl acrylate and acrylic acid (INCI name: Acrylates/Acrylamide Copolymer) are obtainable e.g. as the commercial products Uftrahold Strong® and Ultrahold 8® (BASF).

Polyvinylpyrrolidones (INCI name: PVP) are obtainable e.g. under the trade names Luviskol®K, Luviskol®K30 (BASF) and PVP K® (ISP).

Polyvinylcaprolactams (INCI: Polyvinylcaprolactams) are obtainable e.g. under the trade name Luviskol Plus® (BASF).

Polyurethanes (INCI: Polyurethane-1) are obtainable e.g. under the trade name Luviset®PUR.

Copolymers of acrylic acid, methyl methacrylate, octylacrylamide, butylaminoethyl methacrylate and hydroxypropyl methacrylate (INCI: Octylacrylamide/Acrylates/Butylaminoethyl Methacrylate Copolymer) are known e.g. under the trade names Amphomer®284-910 and Amphomer®LV-71 (National Starch).

Copolymers of vinyl acetate and crotonic acid (INCI: VA/Crotonate Copolymer) are obtainable e.g. under the trade names Luviset®CA 66 (BASF), Resyn®28-1310 (National Starch), Gafset® (GAF) or Aristoflex®A (Celanese).

Copolymers of vinyl acetate, crotonic acid and (vinyl) neodecanoate (INCI: VA/Crotonates/Neodecanoate Copolymer) are obtainable e.g. under the trade names Resyn®28-2930 (National Starch) and Luviset®CAN (BASF).

Copolymers of vinyl acetate and N-vinylpyrrolidone (INCI: PVP/VA) are obtainable e.g. under the trade names Luviskol VA® (BASF) and PVP/VA (ISP).

Carboxy-functional copolymers of vinylpyrrolidone, t-butyl acrylate and methacrylic acid are obtainable e.g. under the trade name Luviskol®VBM (BASF).

Copolymers of tert-butyl acrylate, methacrylic acid and Dimethicone Copolyol are obtainable e.g. under the trade name Luviflex®Silk (BASF).

Suitable anionic polymers are the following, which differ from the copolymers according to the invention: homopolymers and copolymers of acrylic acid and methacrylic acid or salts thereof copolymers of acrylic acid and acrylamide and salts thereof, sodium salts of polyhydroxycarboxylic acids, copolymers of acrylic acid and methacrylic acid with e.g. hydrophobic monomers, examples being C₄-C₃₀-alkyl esters of (meth)acrylic acid, C₄-C₃₀-alkyl vinyl esters, C₄-C₃₀-alkyl vinyl ethers and hyaluronic acid, as well as other polymers known under the trade-names Amerhold®DR-25, Ultrahold®, Luviset®P.U.R., Acronal®, Acudyne®, Lovocryl®, Versatyl®, Arriphomer®(284910, LV-71), Placise®L53, Gantrez®ES 425, Advantage Plus®, Omnirez®2000, Resyn®28-1:310, Resyn®28-2930, Balance®(0/55), Acudyne®255, Aristoflex®A or Eastman AQ®.

The group of suitable polymers further includes e.g. Balance®CR (National Starch), Balance®47 (National Starch; octylacrylamide/acrylate/butylaminoethyl methacrylate copolymer), Aquaflex®FX 64 (ISP; isobutylene/ethylmaleimide/hydroxyethylmaleimide copolymer), Aquaflex®SF40 (ISP/National Starch; VP/vinylcaprolactam/DMAPA acrylate copolymer), Allianz®LT-120 (ISP/Rohm & Haas; acrylate/C₁₋₂ succinate/hydroxyacrylate copolymer) and Aquarez® HS (Eastman; Polyester-1).

Other suitable polymers are those with the trade names Diaformer®Z-400 (Clariant; methacryloyiethylbetaine/methacrylate copolymer), Diaformer®Z-711 (Clariant; methacryloylethyl N-oxide/methacrylate copolymer), Diaformer®Z-712 (Clariant; methacryloylethyl N-oxide/methacrylate copolymer), Omnirez®2000 (ISP; monoethyl ester of poly(methyl vinyl ether/maleic acid) in ethanol), Amphomer®HC (National Starch; acrylate/octylacrylamide copolymer), Amphomer®28-4910 (National Starch; octylacrylamide/acrylate/butylaminoethyl methacrylate copolymer), Advantage®HC 37 (ISP; Terpolymer of vinylcaprolactam/vinylpyrrolidone/dimethylaminoethyl methacrylate), Advantage®LC55 and LC80 or LC A and LC E, Advantage®Plus (ISP; VA/butyl maleate/isobornyl acrylate copolymer), Aculyne®258 (Rohm & Haas; acrylate/hydroxyesteracrylate copolymer), Luviset®P. U. R. (BASF; Polyurethane-1), Eastman®AQ 48 (Eastman), Styleze®CC-10 (ISP; VP/DMAPA/acrylates copolymer), Styleze® 2000 (ISP; VP/acrylates/lauryl methacrylate copolymer), DynamX® (National Starch; Polyurethane-14/AMP-acrylates copolymer), Resyn®XP (National Starch; acrylates/octylacrylamide copolymer), Fixomer® A-30 (Ondeo Nalco; polymethacrylic acid (and) acrylamidomethylpropanesulfonic acid) and Fixate® G-100 (Noveon; AMP-acrylates/allyl methacrylate copolymer).

Other suitable polymers are copolymers of (meth)acrylic acid and polyetheracrylates, the polyether chain terminating with a C₈-C₃₀-alkyl radical. These include e.g. acrylate/beheneth-25 methacrylate copolymers, which are obtainable under the name Aculyn® (Rohm & Haas). Other particularly suitable polymers are copolymers of t-butyl acrylate, ethyl acrylate and methacrylic acid (e.g. Luvimer®100P, Luvimer®Pro55) and copolymers of ethyl acrylate and methacrylic acid. (e.g. Luvimer®MAE).

Crosslinked acrylic acid polymers, such as those obtainable under the INCI name Carbomer, are also suitable. Such crosslinked acrylic acid homopolymers are commercially available e.g. as Carbopol® (Noveon). Hydrophobically modified, crosslinked polyacrylate polymers, such as Carbopol®Ultrez 21 (Noveon), are also preferred. Said other polymers can also be used to modify the rheology of the compositions, i.e. as thickeners.

Other suitable additional polymers are water-soluble or water-dispersible polyesters, polyureas, polyurethanes, polyurethaneureas, maleic anhydride copolymers which have optionally been reacted with alcohols, or anionic polysiloxanes.

Other polymers suitable for use together with the polymers A are e.g. cationic and cationogenic polymers. These include e.g.
copolymers of N-vinylpyrrolidone/N-vinylimidazolium salts (obtainable e.g. under the trade names Luviquat®FC, Luviquat®HM, Luviquat®MS, Luviquat®Care and Luviquat® UltraCare (BASF)),
quaternized copolymers of vinyl pyrrolidone, methacrylamide, vinylimidazole and quaternized vinylimidazole (obtainable e.g. under the trade name Luviquat®Supreme),
copolymers of N-vinylcaprolactam/N-vinylpyrrolidone/N-vinylimidazolium salts (obtainable e.g. under the trade name Luviquat®Hold),
copolymers of N-vinylpyrrolidone/dimethylaminoethyl methacryleate quaternized with diethyl sulfate (obtainable e.g. under the trade name Luviquat®PQ11),
copolymers of vinylpyrrolidone, methacrylamide and vinylimidazole (Luviset®Clear),
cationic cellulose derivatives (Polyquaternium-4 and -10),
acrylamide copolymers (Polyquaternium-7),
guar hydroxypropyltrimethylammonium chloride (INCI: Hydroxypropyl Guar Hydroxypropyltrimonium Chloride),
polyethylene imines and salts thereof,
polyvinylamines and salts thereof,
polymers based on dimethyldiallylammonium chloride (Merquat®),
polymers formed by reacting polyvinylpyrrolidone with quaternary ammonium compounds (Gafquat®),
hydroxyethyl cellulose with cationic groups (Polymer®JR), and
plant-based cationic polymers, e.g. guar polymers such as the Jaguar® marks from Rhodia.

Other suitable cosmetic polymers for the hair are neutral polymers such as
polyvinylpyrrolidones,
copolymers of N-vinylpyrrolidone and vinyl acetate and/or vinyl propionate,
polysiloxanes,
polyvinylcaprolactams,
copolymers with N-vinylpyrrolidone,
cellulose derivatives,
polyaspartic acid salts and derivatives, and
polyamides based e.g. on itaconic acid and aliphatic diamines, such as those described in DE-A-43 33 238.

The aforesaid types of polymers include those known under the trade names Luviskol® (K, VA, Plus), PVP K, PVPNA, Advantage®HC, Luviflex®Swing, Kollicoat®IR and H₂OLD®EP-1.

Other suitable polymers are biopolymers, i.e. polymers obtained from naturally growing raw materials and built up of natural monomeric structural units, e.g. derivatives of cellulose, chitin, chitosan, DNA, hyaluronic acid and RNA.

Other suitable mixing partners for the polymers according to the invention are zwitterionic polymers such as those disclosed e.g. in German patent applications DE 39 29 973, DE 21 50 557, DE 28 17 369 and DE 37 08 451, the methacryloyl-ethylbetaine/methacrylate copolymers commercially available under the name Amersette® (Amerchol), or copolymers of hydroxyethyl methacrylate, methyl methacrylate, N,N-dimethylaminoethyl methacrylate and acrylic acid (Jordapon®).

Other suitable polymers are betaine polymers such as Yukaformer (R205, SM) and Diaformer.

Other polymers suitable as mixing partners are non-ionic, siloxane-containing, water-soluble or water-dispersible polymers, e.g. polyethersiloxanes such as Tegopren® (Goldschmidt) or Belsil® (Wacker).

Cosmetically and/or Dermatologically Active Substances

Examples of suitable cosmetically and/or dermatologically active substances are dyestuffs, skin and hair pigmenting agents, toners, browning ingredients, bleaching agents, keratin-hardening substances, antimicrobial substances, light filters, repellents, substances with a hyperemic action, substances with a keratolytic and keratoplastic action, antidandruff substances, antiphlogistics, substances with a keratinizing action, substances active as antioxidants or free radical scavengers, skin moisturizers or humectants, superfatting substances, substances with an antierythematous or antiallergic action, and mixtures thereof.

Preferred cosmetic nurturing substances and active substances are AHA acids, fruit acids, ceramides, phytantriol, collagen, vitamins and provitamins, e.g. vitamins A, E and C, retinol, bisabolol and panthenol. A particularly preferred cosmetic nurturing substance in the compositions according to the invention is panthenol, which is commercially available e.g. as D-Panthenol®USP, D-Panthenol®50 P, D-Panthenol®75 W and D,L-Panthenol®50 W.

Examples of artificial skin-browning substances that are suitable for browning the skin without natural or artificial UV irradiation are dihydroxyacetone, alloxan and walnut shell extract.

Suitable keratin-hardening substances are normally the active substances also used in antiperspirants, e.g. potassium aluminum sulfate, aluminum hydroxychloride, aluminum lactate, etc.

Antimicrobial substances are used to destroy microorganisms or inhibit their growth and thus serve both as preservatives and as deodorizing substances that reduce the occurrence or intensity of body odor. They include e.g. conventional preservatives known to those skilled in the art, such as p-hydroxybenzoic acid esters, imidazolidinyl-urea, formaldehyde, sorbic acid, benzoic acid, salicylic acid, etc. Examples of such deodorizing substances are zinc ricinoleate, triclosan, undecylenic acid alkylolamides, triethyl citrate, chlorhexidine, etc. The compositions according to the invention preferably contain 0.01 to 5 and particularly preferably 0.05 to 1% by weight of at least one preservative. Other suitable preservatives are the substances listed under the function “Preservatives” in the International Cosmetic Ingredient Dictionary and Handbook, 9th edition, e.g. phenoxyethanol, benzylparaben, butylparaben, ethylparaben, isobutylparaben, isopropylparaben, methylparaben, propylparaben, iodopropynylbutyl carbamate, methyldibromoglutaronitrile and DMDM hydantoin.

### UV Filters

In one embodiment the compositions according to the invention can contain oil-soluble and/or water-soluble UVA and/or UVB filters.

The total amount of filters is preferably 0.01 to 10% by weight or from 0.1 to 5% by weight and particularly preferably from 0.2 to 2% by weight, based on the total weight of the compositions.

The major part of the sunscreen agents in the compositions used to protect the human epidermis consists of compounds that absorb UV light in the UVB region. For example, the proportion of UVA absorbers to be used according to the invention is 10 to 90% by weight and preferably 20 to 50% by weight, based on the total amount of UVB- and UVA-absorbing substances.

The UVB filters can be oil-soluble or water-soluble. Examples of advantageous UVB filters are:
i) benzimidazolesulfonic acid derivatives, e.g. 2-phenylbenzimidazole-5-sulfonic acid and its salts;
ii) benzotriazole derivatives, e.g. 2,2′-methylenebis(6-(2H-benzotriazol-2-yl)-4-(1,1,3,3-tetramethylbutyl)phenol);
iii) 4-aminobenzoic acid derivatives, preferably 2-ethylhexyl 4-(dimethylamino)-benzoate and amyl 4-(dimethylamino)benzoate;
iv) benzalmalonic acid esters, preferably di(2-ethylhexyl) 4-methoxybenzal-malonate;
v) cinnamic acid esters, preferably 2-ethylhexyl 4-methoxycinnamate and isopentyl 4-methoxycinnamate;
vi) benzophenone derivatives, preferably 2-hydroxy-4-methoxybenzophenone, 2-hydroxy-4-methoxy-4′-methylbenzophenone and 2,2′-dihydroxy-4-methoxy-benzophenone;
vii) methylidenecamphor derivatives, preferably 4-methylbenzylidenecamphor and benzylidenecamphor; and
viii) triazine derivatives, preferably tris(2-ethylhexyl) 4,4′,4″-(1,3,5-triazin-2,4,6-triylimino)trisbenzoate [INCI: Diethylhexyl Butamido Triazine, UVA-Sorb® HEB (Sigma 3V)] and 2,4,6-tris[anilino(p-carbo-2′-ethyl-1′-hexyloxy)]-1,3,5-triazine [INCI: Octyl Triazone, Uvinul®T 150 (BASF)].

Examples of water-soluble UVB filters which can advantageously be used are sulfonic acid derivatives of 3-benzylidenecamphor, such as 4-(2-oxo-3-bornylidenemethyl)-benzenesulfonic acid, 2-methyl-5-(2-oxo-3-bornylidenemethyl)sulfonic acid and salts thereof.

Examples of UVA filters which can advantageously be used are:
i) 1,4-phenylenedimethynecamphorsulfonic acid derivatives, e.g. 3,3′-(1,4-phenylenedimethyne)bis(7,7-dimethyl-2-oxobicyclo[2.2.1]heptane-1-methane-sulfonic acid) and its salts;
ii) 1,3,5-triazine derivatives such as 2,4-bis{[(2-ethylhexyloxy)-2-hydroxy]phenyl}-6-(4-methoxyphenyl)-1,3,5-triazine (e.g. Tinosorb®S (Ciba));
iii) dibenzoylmethane derivatives, preferably 4-isopropyldibenzoylmethane and 4-(tert-butyl)-4′-methoxydibenzoylmethane;
iv) benzoxazole derivates, e.g. 2,4-bis[4-[5-(1,1-dimethylpropyl)benzoxazol-2-yl]-phenylimino]-6-[(2-ethylhexyl)imino]-1,3,5-triazine (CAS No. 288254-1 6-0, Uvasorb®K2A (3V Sigma)); and
v) hydroxybenzophenones, e.g. hexyl 2-(4′-diethylamino-2′-hydroxybenzoyl)-benzoate (also: aminobenzophenone) (Uvinul®A Plus (BASF)).

Furthermore, it can optionally be advantageous according to the invention to incorporate other UVA and/or UVB filters into the compositions, examples being specific salicylic acid derivatives such as 4-isopropylbenzyl salicylate, 2-ethylhexyl salicylate, octyl salicylate and homomethyl salicylate. The total amount of salicylic acid derivatives in the cosmetic compositions is advantageously chosen within the range of 0.1-15.0 and preferably of 0.3-10.0% by weight, based on the total weight of the compositions. Another light filter which can advantageously be used according to the invention is ethylhexyl 2-cyano-3,3-diphenylacrylate (octocrylene, Uvinul®N 539 (BASF)).

Some light filters suitable for use in the compositions according to the invention are collated by way of example in the Table below:

| No. | Substance | CAS No. |
|---|---|---|
| 1 | 4-Aminobenzoic acid | 150-13-0 |
| 2 | 3-(4′-Trimethylammonium)benzylidenebornan-2-one | 52793-97-2 |
| | methylsulfate | |
| 3 | 3,3,5-Trimethylcyclohexyl salicylate | 118-56-9 |
| | (Homosalatum) | |
| 4 | 2-Hydroxy-4-methoxybenzophenone | 131-57-7 |
| | (Oxybenzonum) | |
| 5 | 2-Phenylbenzimidazole-5-sulfonic acid and its potassium, | 27503-81-7 |
| | sodium and triethanolamine salts | |
| 6 | 3,3′-(1,4-Phenylenedimethyne)bis(7,7-dimethyl- | 90457-82-2 |
| | 2-oxobicyclo[2.2.1]heptane-1-methanesulfonic acid) and its | |
| | salts | |
| 7 | Polyethoxyethyl 4-bis(polyethoxy)aminobenzoate | 113010-52-9 |
| 8 | 2-Ethylhexyl 4-dimethylaminobenzoate | 21245-02-3 |
| 9 | 2-Ethylhexyl salicylate | 118-60-5 |
| 10 | 2-Isoamyl 4-methoxycinnamate | 71617-10-2 |
| 11 | 2-Ethylhexyl 4-methoxycinnamate | 5466-77-3 |
| 12 | 2-Hydroxy-4-methoxybenzophenone-5-sulfonic acid | 4065-45-6 |
| | (Sulisobenzonum) and the sodium salt | |
| 13 | 3-(4′-Sulfobenzylidene)bornan-2-one and salts | 58030-58-6 |
| 14 | 3-Benzylidenebornan-2-one | 16087-24-8 |
| 15 | 1-(4′-Isopropylphenyl)-3-phenylpropane-1,3-dione | 63260-25-9 |
| 16 | 4-Isopropylbenzyl salicylate | 94134-93-7 |
| 17 | 3-Imidazol-4-ylacrylic acid and its ethyl ester | 104-98-3 |
| 18 | Ethyl 2-cyano-3,3-diphenylacrylate | 5232-99-5 |
| 19 | 2′-Ethylhexyl 2-cyano-3,3-diphenylacrylate | 6197-30-4 |
| 20 | Menthyl o-aminobenzoate or | 134-09-8 |
| | 5-methyl-2-(1-methylethyl)-2-aminobenzoate | |
| 21 | Glyceryl p-aminobenzoate or | 136-44-7 |
| | 1-Glyceryl 4-aminobenzoate | |
| 22 | 2,2′-Dihydroxy-4-methoxybenzophenone (Dioxybenzone) | 131-53-3 |
| 23 | 2-Hydroxy-4-methoxy-4-methylbenzophenone | 1641-17-4 |
| | (Mexenone) | |
| 24 | Triethanolamine salicylate | 2174-16-5 |
| 25 | Dimethoxyphenylglyoxalic acid or | 4732-70-1 |
| | Sodium 3,4-dimethoxyphenylglyoxalate | |
| 26 | 3-(4′-Sulfobenzylidene)bornan-2-one and its salts | 56039-58-8 |
| 27 | 4-tert-Butyl-4′-methoxydibenzoylmethane | 70356-09-1 |
| 28 | 2,2′,4,4′-Tetrahydroxybenzophenone | 131-55-5 |
| 29 | 2,2′-Methylenebis[6-(2H-benzotriazol-2-yl)-4-(1,1,3,3- | 103597-45-1 |
| | tetramethylbutyl)phenol] | |
| 30 | 2,2′-(1,4-Phenylene)bis-1H-benzimidazole-4,6- | 180898-37-7 |
| | disulfonic acid, Na salt | |
| 31 | 2,4-bis[4-(2-Ethylhexyloxy)-2-hydroxy]phenyl- | 187393-00-6 |
| | 6-(4-methoxyphenyl)-1,3,5-triazine | |
| 32 | 3-(4-Methylbenzylidene)camphor | 36861-47-9 |
| 33 | Polyethoxyethyl 4-bis(polyethoxy)paraaminobenzoate | 113010-52-9 |
| 34 | 2,4-Dihydroxybenzophenone | 131-56-6 |
| 35 | 2,2′-Dihydroxy-4,4′-dimethoxybenzophenone-5,5′- | 3121-60-6 |
| | sulfonate, disodium salt | |
| 36 | 2-[4-(Diethylamino)-2-hydroxybenzoyl]hexyl benzoate | 302776-68-7 |
| 37 | 2-(2H-Benzotriazol-2-yl)-4-methyl-6-[2-methyl-3-[1,3,3,3- | 155633-54-8 |
| | tetramethyl-1-[(trimethylsilyl)oxy]disiloxanyl]propyl]phenol | |
| 38 | 1,1-[(2,2′-Dimethylpropoxy)carbonyl]-4,4-diphenyl-1,3-butadiene | 363602-15-7 |

Suitable UV filters of CAS No. 113010-52-9 are commercially available e.g. under the name Uvinul ® P 25.
Polymeric or polymer-bonded filters can also be used according to the invention.

Metal oxides, such as titanium dioxide or zinc oxide, can likewise advantageously be used to protect against harmful solar radiation. Their action is based essentially on reflection, dispersion and absorption of the harmful UV radiation and depends substantially on the primary particle size of the metal oxides. The cosmetic compositions according to the invention can also advantageously contain inorganic pigments based on metal oxides and/or other metal compounds sparingly soluble or insoluble in water, selected from the group comprising the oxides of zinc (ZnO), iron (e.g. Fe₂O₃), zirconium (ZrO₂), silicon (SiO₂), manganese (e.g. MnO), aluminum (Al₂O₃) and cerium (e.g. Ce₂O₃), mixed oxides of said metals, and mixtures of such oxides. ZnO-based pigments are particularly preferred.

The inorganic pigments can be present in coated form, i.e. they are surface-treated. This surface treatment can consist e.g. in providing the pigments with a thin hydrophobic layer in a manner known per se, as described in DE-A-33 14 742. Sunscreen agents suitable for use in the compositions according to the invention are the compounds mentioned in paragraphs [0036] to [0053] of EP-A 1 084 696, which is incorporated here to the full extent by way of reference. All the UV filters mentioned under “Ultraviolett-Filter für kosmetische Mittel” (“Ultraviolet Filters for Cosmetic Compositions”) in Annex 7 (under § 3b) of the German Cosmetics Directive are suitable for use according to the invention.

The given list of UV filters which can be used in the compositions according to the invention is not comprehensive.

### Thickeners

Suitable thickeners are mentioned in “Kosmetik und Hygiene von Kopf bis Fuβ” (“Cosmetics and Hygiene from Head to Toe”), edited by W. Umbach, 3rd edition, Wiley-VCH, 2004, pp 235-236, which is incorporated here to the full extent by way of reference.

The viscosity of e.g. shampoos can be adjusted to the desired value with texture regulators. Thickeners, which have a viscosity-enhancing effect by enlarging the surfactant micelles or swelling the aqueous phase, derive from chemically very different classes of substances.

Suitable thickeners for the compositions according to the invention are crosslinked polyacrylic acids and derivatives thereof, polysaccharides such as xanthan gum, guar-guar, agar-agar, alginates or tyloses, cellulose derivatives, e.g. carboxymethyl cellulose or hydroxycarboxymethyl cellulose, higher-molecular polyethylene glycol monoesters and diesters of fatty acids, and fatty alcohols, monoglycerides, fatty acids, polyvinyl alcohol and polyvinylpyrrolidone.

Other suitable thickeners are polyacrylates such as Carbopol® (Noveon), Ultrez® (Noveon), Luvigel® EM (BASF), Capigel®98 (Seppic), Synthalene® (Sigma) and the Aculyn® marks from Rohm and Haas, such as Aculyn® 22 (copolymer of acrylates and methacrylic acid ethoxylates with a stearyl radical (20 EO units)) and Aculyn® 28 (copolymer of acrylates and methacrylic acid ethoxylates with a behenyl radical (25 EO units)).

Examples of other suitable thickeners are Aerosil grades (hydrophilic silicic acids), polyacrylamides, polyvinyl alcohol and polyvinylpyrrolidone, surfactants, e.g. ethoxylated fatty acid glycerides, esters of fatty acids with polyols such as pentaerythritol or trimethylolpropane, fatty alcohol ethoxylates with a narrow homolog distribution, or alkyloligoglucosides, and electrolytes such as sodium chloride and ammonium chloride.

Particularly preferred thickeners for the preparation of gels are Ultrez®21, Aculyn®28, Luvigel® EM and Capigel®98.

Particularly in the case of more highly concentrated shampoo formulations, the texture can also be regulated by adding substances which reduce the viscosity of the formulation, e.g. propylene glycol or glycerol. These substances have little effect on the product properties.

### Gelling Agents

If it is desired, to use gelling agents for the compositions according to the invention, any of the gelling agents conventionally used in cosmetics can be employed. These include slightly crosslinked polyacrylic acid, e.g. Carbomer (INCI), cellulose derivatives, e.g. hydroxypropyl cellulose, hydroxyethyl cellulose and cationically modified celluloses, polysaccharides, e.g. Xanthum Gummi, Caprylic/Capric Triglyceride, Sodium Acrylates Copolymer, Polyquaternium-32 (and) Paraffinum Liquidum (INCI), Sodium Acrylates Copolymer (and) Paraffinum Liquidum (and) PPG-1 Trideceth-61 Acrylamidopropyl Trimonium Chloride/Acrylamide Copolymer, Steareth-10 Allyl Ether Acrylates Copolymer, Polyquaternium-37 (and) Paraffinum Liquidum (and) PPG-1 Trideceth-6, Polyquaternium-37 (and) Propylene Glycol Dicaprate Dicaprylate (and) PPG-1 Trideceth-6, Polyquaternium-7 and Polyquaternium-44.

### Emulsifiers

Examples of suitable emulsifiers are non-ionogenic surfactants from at least one of the following groups:
i) addition products of 2 to 30 mol of ethylene oxide and/or 0 to 5 mol of propylene oxide with linear fatty alcohols having 8 to 22 C atoms, with fatty acids having 12 to 22 C atoms and with alkylphenols having 8 to 15 C atoms in the alkyl group;
ii) C12/18-fatty acid monoesters and diesters of addition products of 1 to 30 mol of ethylene oxide with glycerol;
iii) glycerol monoesters and diesters and sorbitan monoesters and diesters of saturated and unsaturated fatty acids having 6 to 22 carbon atoms, and ethylene oxide addition products thereof;
iv) alkylmonoglycosides and alkyloligoglycosides having 8 to 22 carbon atoms in the alkyl radical, and ethoxylated analogs thereof;
v) addition products of 15 to 60 mol of ethylene oxide with castor oil and/or hydrogenated castor oil;
vi) polyol and, in particular, polyglycerol esters, e.g. polyglycerol polyricinoleate, polyglycerol poly-12-hydroxystearate or polyglycerol dimerate; mixtures of compounds from several of these classes of substances are also suitable;
vii) addition products of 2 to 15 mol of ethylene oxide with castor oil and/or hydrogenated castor oil;
viii) partial esters based on linear or branched, unsaturated or saturated C_{6/22}-fatty acids, ricinoleic acid and 12-hydroxystearic acid with glycerol, polyglycerol, pentaerythritol, dipentaerythritol, sugar alcohols (e.g. sorbitol), alkylglucosides (e.g. methylglucoside, butylglucoside, laurylglucoside) and polyglucosides (e.g. cellulose);
ix) mono-, di- and trialkyl phosphates and mono-, di- and/or tri-PEG-alkyl phosphates and salts thereof;
x) lanolin alcohols;
xi) polysiloxane-polyalkyl-polyether copolymers or corresponding derivatives;
xii) mixed esters of pentaerythritol, fatty acids, citric acid and fatty alcohol according to DE-PS 1165574, and/or mixed esters of fatty acids having 6 to 22 carbon atoms, methylglycose and polyols, preferably glycerol or polyglycerol; and
xiii) polyalkylene glycols.

The addition products of ethylene oxide and/or propylene oxide with fatty alcohols, fatty acids, alkylphenols, glycerol monoesters and diesters and sorbitan monoesters and diesters of fatty acids, or with castor oil, are known, commercially available products. They are homolog mixtures whose mean degree of alkoxylation corresponds to the ratio of the amounts of ethylene Oxide and/or propylene oxide and substrate with which the addition reaction is carried out. C₁₂- to C₁₈-fatty acid monoesters and diesters of addition products of ethylene oxide with glycerol are known from DE-PS 2024051 as superfatting agents for cosmetic compositions. C₈- to C₁₈-alkylmonoglycosides and -alkyloligoglycosides, their preparation and their use are known from the state of the art. They are prepared in particular by reacting glucose or oligosaccharides with primary alcohols having 8 to 18 C atoms. As far as the glycoside ester is concerned, both monoglycosides in which a cyclic sugar residue is glycosidically linked to the fatty alcohol, and also oligomeric glycosides with a degree of oligomerization preferably of up to about 8, are suitable. The degree of oligomerization here is a statistical mean based on a homolog distribution conventionally used for such industrial products.

Zwitterionic surfactants can also be used as emulsifiers. Zwitterionic surfactants are understood as meaning surface-active compounds carrying at least one quaternary ammonium group and at least one carboxylate and/or sulfonate group per molecule.

Particularly suitable zwitterionic surfactants are the so-called betaines such as N-alkyl-N,N-dimethylammonium glycinates, e.g. cocoalkyldimethylammonium glycinate, N-acylaminopropyl-N,N-dimethylammonium glycinates, e.g. cocoacylaminopropyl-dimethylammonium glycinate, and 2-alkyl-3-carboxymethyl-3-hydroxyethyl-imidazolines, each having 8 to 18-C atoms in the alkyl or acyl group, and cocoacyl-aminoethylhydroxyethylcarboxymethylglycinate.

The fatty acid amide derivative known by the CTFA name Cocamidopropyl Betaine is particularly preferred. Other suitable emulsifiers are ampholytic surfactants. Ampholytic surfactants are understood as meaning surface-active compounds which, apart from a C₈- to C₁₈-alkyl or -acyl group, contain at least one free amino group and at least one —COOH and/or —SO₃H group per molecule and are capable of forcing, internal salts. Examples of suitable ampholytic-surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids each having about 8 to 18 C atoms in the alkyl group. Particularly preferred ampholytic surfactants are N-cocoalkylaminopropionate, cocoacylaminoethylaminopropionate and C₁₂- to C₁₈-acylsarcosine.

Other possible emulsifiers apart from ampholytic emulsifiers are quaternary emulsifiers, particular preference being given to those of the Esterquat type, preferably methyl-quaternized difatty acid triethanolamine ester salts.

### Antioxidants

It can be advantageous for the compositions additionally to contain antioxidants. According to the invention, any of the antioxidants that are suitable or customary for cosmetic applications can be used. The antioxidants are advantageously selected from the group comprising amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotinoids, carotinenes (e.g. α-carotene, β-carotene, γ-lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and their glycosyl, N-acetyl, methyl, ethyl, propyl, amyl, butyl, lauryl, palmitoyl, oleyl, γ-linoleyl, cholesteryl and glyceryl esters) and salts thereof, dilauryl thiodipropionate, distearyl thiodipropionate, thiodipropionic acid and derivatives thereof (esters, ethers, peptides, lipids, nucleotides, nucleosides and salts), sulfoximine compounds (e.g. buthionine sulfoximines, homocysteine sulfoximine, buthionine sulfones, penta-, hexa- and heptathionine sulfoximine) in very low compatible dosages (e.g. pmol to μmol/kg), (metal) chelators (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, furfurylidenesorbitol and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), coniferyl benzoate from benzoin, rutic acid and derivatives thereof, α-glycosylrutin, ferulic acid, furfurylidene-glucitol, carnosine, butylhydroxytoluene, butylhydroxyanisole, nordihydroguaiacic acid, nordihydroguaiaretic acid, trihydroxybutyrophenone, uric acid and derivatives thereof mannose and derivatives thereof, zinc and derivatives-thereof (e.g. ZnO, ZnSO₄), selenium and derivatives thereof (e.g. selenium methionine), stilbenes and derivatives thereof (e.g. stilbene oxide, trans-stilbene oxide), and the derivatives suitable according to the invention (salts, esters, ethers, sugars, nucleotides, nucleosides, peptides and lipids) of said active substances.

The amount of the aforesaid antioxidants (one or more compounds) in the compositions is preferably 0.001 to 30% by weight, particularly preferably 0.05 to 20% by weight and very particularly preferably 0.1 to 10% by weight, based on the total weight of the composition.

If the antioxidant or antioxidants are represented by vitamin E and/or derivatives thereof, it is advantageous to provide them in concentrations of 0.001 to 10% by weight, based on the total weight of the composition.

If the antioxidant or antioxidants are represented by vitamin A or vitamin A derivatives or by carotenes or derivatives thereof; it is advantageous to provide them in concentrations of 0.001 to 10% by weight, based on the total weight of the composition.

### Perfume Oils

The cosmetic compositions, preferably for the hair, can contain perfume oils. Mixtures of natural and synthetic perfumes may be mentioned as examples of perfume oils.

Natural perfumes are extracts of blossoms (lily, lavender, rose, jasmine, neroli, ylang-ylang), stems and leaves (geranium, patchouli, petitgrain), fruits (anise, coriander, caraway, juniper), fruit peels (bergamot, lemon, orange), roots (mace, angelica, celery, cardamom, costus, iris, calamus), woods (pinewood, sandalwood, guaiac wood, cedarwood, rosewood), herbs and grasses (tarragon, lemongrass, sage, thyme), needles and twigs (spruce, fir, pine (Pinus), pine (Pinus montana)) and resins and balsams (galbanum, elemi, benzoin, myrrh, frankincense, opoponax). Animal raw materials are also suitable, examples being civet and castoreum. Typical synthetic perfume compounds are products of the ester, ether, aldehyde, ketone, alcohol and hydrocarbon type. Examples of perfume compounds of the ester type are benzyl acetate, phenoxyethyl isobutyrate, 4-tert-butylcyclohexyl acetate, linalyl acetate, dimethylbenzylcarbinyl acetate, phenylethyl acetate, linalyl benzoate, benzyl formate, ethylmethylphenyl glycinate, allylcyclohexyl propionate, styrallyl propionate and benzyl salicylate. The ethers include e.g. benzyl ethyl ether, the aldehydes include e.g. linear alkanals having 8 to 18 carbon atoms, citral, citronellal, citronellyloxyacetaldehyde, cyclamen aldehyde, hydroxycitronellal, lilial and bourgeonal, the ketones include e.g. ionones, cc-isomethylionene and methyl cedryl ketone, the alcohols include anethole, citronellol, eugehol, isoeugenol, geranioli linalool, phenylethyl alcohol and terioneol, and the hydrocarbons include principally terpenes and balsams. It is preferable, however, to use mixtures of different perfumes which together produce an appealing scent. Ethereal oils of lower volatility, which are generally used as flavoring components, are also suitable as perfume oils, examples being sage oil, camomile oil, clove oil, melissa oil, mint oil, cinnamon leaf oil, lime blossom oil, juniper berry oil, vetiver oil, oliban oil, galbanum oil, labolanum oil and lavandin oil. It is preferable to use bergamot oil, dihydromyrcenol, lilial, lyral, citronellol, phenylethyl alcohol, α-hexylcinnamaldehyde, geraniol, benzylacetone, cyclamen aldehyde, linalool, boisambrene forte, ambroxane, indole, hedione, sandelice, lemon oil, mandarin oil, orange oil, allylamyl glycolate, cyclovertal, lavandin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romilat, irotyl and floramat, by themselves or in mixtures.

### Hyperfatting Agents

Hyperfatting agents which can be used are substances such as lanolin and lecithin, as well as polyethoxylated or acylated lanolin and lecithin derivatives, polyol fatty acid esters, monoglycerides and fatty acid alkanolamides, the latter simultaneously serving as foam stabilizers.

### Silicone Compounds

In one embodiment the compositions according to the invention contain, as a hair nurturing additive, at least one silicone compound in an amount preferably of 0.01 to 15% by weight and particularly preferably of 0.1 to 5% by weight. The silicone compounds comprise volatile and non-volatile silicones and silicones that are soluble and insoluble in the composition. In one embodiment these silicone compounds are high-molecular silicones having a viscosity of 1000 to 2,000,000 cSt at 25° C., and preferably of 10,000 to 1,800,000 or 100,000 to 1,500,000. The silicone compounds comprise polyalkylsiloxanes and polyarylsiloxanes, especially those having methyl, ethyl, propyl, phenyl, methylphenyl and phenylmethyl groups. Polydimethylsiloxanes, polydiethylsiloxanes and polymethylphenylsiloxanes are preferred. Arylated silicones which confer luster, having a refractive index of at least 1.46or at least 1.52, are also preferred. The silicone compounds comprise especially the substances with the INCI names Cyclomethicone, Dimethicone, Dimethiconol, Dimethicone Copolyol, Phenyl Trimethicone, Amodimethicone, Trimethylsilylamodimethicone, Stearyl Siloxysilicate, Polymethylsilsesquioxane and Dimethicone Crosspolymer. Silicone resins and silicone elastomers, i.e. highly crosslinked siloxanes, are also suitable.

Preferred silicones are cyclic dimethylsiloxanes, linear polydimethylsiloxanes, block polymers of polydimethylsiloxane and polyethylene oxide and/or polypropylene oxide, polydimethylsiloxanes with terminal or lateral polyethylene oxide or polypropylene oxide radicals, polydimethylsiloxanes with terminal hydroxyl groups, phenyl-substituted polydimethylsiloxanes, silicone emulsions, silicone elastomers, silicone waxes, silicone gums and amino-substituted silicones (CTFA: Amodimethicone).

### Hair Conditioners

In one embodiment the compositions according to the invention contain 0.01 to 20, preferably from 0.05 to 10 and particularly preferably from 0.1 to 5% by weight of at least one conditioner.

Examples of conditioners which are preferred according to the invention are any compounds listed under Section 4 of the International Cosmetic Ingredient Dictionary and Handbook (volume 4, editors: R. C. Pepe, J. A. Wenninger, G. N. McEwen, The Cosmetic, Toiletry and Fragrance Association, 9th edition, 2002) under the keywords Hair-Conditioning Agents, Humectants, Skin-Conditioning Agents, Skin-Conditioning Agents-Emollient, Skin-Conditioning Agents-Humectant, Skin-Conditioning Agents-Miscellaneous, Skin-Conditioning Agents-Occlusive and Skin Protectants, and any compounds listed in EP-A 934 956 (pp 11-13) under “water soluble conditioning agent” and “oil soluble conditioning agent”. Examples of other advantageous conditioners are the compounds with the INCI name Polyquaternium (especially Polyquaternium-1 to Polyquaternium-56).

Suitable conditioners also include e.g. polymeric quaternary ammonium compounds, cationic cellulose derivatives, chitosan derivatives and polysaccharides.

The conditioner is preferably selected from betaine, panthenol, panthenyl ethyl ether, sorbitol, protein hydrolyzates and plant extracts; A-B block copolymers of alkyl acrylates and alkyl methacrylates; A-B block copolymers of alkyl methacrylates and acrylonitrile; A-B-A block copolymers of lactide and ethylene oxide; A-B-A block copolymers of caprolactone and ethylene oxide; A-B-C block copolymers of alkylene or alkadiene compounds, styrene and alkyl methacrylates; A-B-C block copolymers of acrylic acid, styrene and alkyl methacrylates; stellate block copolymers; hyper-branched polymers; dendrimers; intrinsically electrically conducting 3,4-pblyethylene-dioxythiophenes; and intrinsically electrically conducting polyanilines.

Other conditioners which are advantageous according to the invention are cellulose derivatives and quaternized guar gum derivatives, especially guar hydroxypropyl-ammonium chloride (e.g. Jaguar Excel®, Jaguar C 162® (Rhodia), CAS 65497-29-2, CAS 39421-75-5).

Non-ionic poly-N-vinylpyrrolidone/polyvinyl acetate copolymers (e.g. Luviskol®VA 64 (BASF)), anionic acrylate copolymers (e.g. Luviflex®Soft (BASF)) and/or amphoteric amide/acrylate/methacrylate copolymers (e.g. Amphomer® (National Starch)) can also be used advantageously according to the invention as conditioners.

### Hydrotropic Agents

The flow behavior can be improved by also using hydrotropic agents, e.g. ethanol, isopropyl alcohol or polyols. Polyols which are suitable here preferably have 2 to 15 carbon atoms and at least two hydroxyl groups. Typical examples are
i) glycerol;
ii) alkylene glycols, e.g. ethylene glycol, diethylene glycol, propylene glycol, butylene glycol, hexylene glycol and polyethylene glycols having an average molecular weight of 100 to 1000 daltons;
iii) technical-grade oligoglycerol mixtures having a degree of autocondensation of 1.5 to 10, for instance technical-grade diglycerol mixtures with a diglycerol content of 40 to 50% by weight;
iv) methylol compounds such as, in particular, trimethylolethane, trimethylol-propane, trimethylolbutane, pentaerythritol and dipentaerythritol;
v) lower alkylglucosides, especially those having 1 to 8 carbon atoms in the alkyl radical, e.g. methylglucoside and butylglucoside;
vi) sugar alcohols having 5 to 12 carbon atoms, e.g. sorbitol or mannitol;
vii) sugars having 5 to 12 carbon atoms, e.g. glucose or sucrose; and
viii) amino sugars, e.g. glucamine.

### Oils, Fats and Waxes

The cosmetic compositions according to the invention, preferably for the hair, can also contain oils, fats or waxes. These are advantageously selected from the group comprising lecithins and fatty acid triglycerides, namely the triglycerol esters of saturated and/or unsaturated, branched and/or unbranched alkanecarboxylic acids having a chain length of 8 to 24 and especially of 12 to 18 C atoms. The fatty acid triglycerides can advantageously be selected e.g. from the group comprising synthetic, semisynthetic and natural oils, e.g. olive oil, sunflower oil, soya oil, groundnut oil, rapeseed oil, almond oil, palm oil, coconut oil, castor oil, wheatgerm oil, grapeseed oil, thistle oil, evening primrose oil, macadamia nut oil and the like. Other polar oil components can be selected from the group comprising the esters of saturated and/or unsaturated, branched and/or unbranched alkanecarboxylic acids having a chain length of 3 to 30 C atoms with saturated and/or unsaturated, branched and/or unbranched alcohols having a chain length of 3 to 30 C atoms, and from the group comprising the esters of aromatic carboxylic acids with saturated and/or unsaturated, branched and/or unbranched alcohols having a chain length of 3 to 30 C atoms. Such ester oils can then advantageously be selected from the group comprising isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palpitate, 2-ethylhexyl laurate, 2-hexyldecyl stearate, 2-octyldodecyl palmitate, oleyl oleate, oleyl erucate, erucyl oleate, erucyl erucate, dicaprylyl carbonate (Cetiol CC) and cocoglycerides (Myritol 331), butylene glycol dicaprylate/dicaprate and dibutyl adipate, as well as synthetic, semisynthetic and natural mixtures of such esters, e.g. jojoba oil.

Furthermore, one or more oil components can advantageously be selected from the group comprising branched and unbranched hydrocarbons and hydrocarbon waxes, silicone oils and dialkyl ethers, and from the group comprising saturated or unsaturated, branched or unbranched alcohols.

Any desired mixtures of such oil and wax components can also advantageously be used within the framework of the present invention. It may also be advantageous to use waxes, e.g. cetyl palmitate, as the only lipid component of the oily phase.

According to the invention, the oil component is advantageously selected from the group comprising 2-ethylhexyl isostearate, octyldodecanol, isotridecyl isononanoate, isoeicosane, 2-ethylhexyl cocoate, C12-15-alkyl benzoate, caprylic/capric triglyceride and dicaprylyl ether.

Mixtures of C12-15-alkyl benzoate and 2-ethylhexyl isostearate, mixtures of C12-15-alkyl benzoate and isotridecyl isononanoate and mixtures of C12-15-alkyl benzoate, 2-ethylhexyl isostearate and isotridecyl isononanoate are advantageous according to the invention.

It is particularly preferable according to the invention to use-fatty acid triglycerides, especially soya oil and/or almond oil, as oils with a polarity of 5 to 50 mN/Nm.

The oily phase can also advantageously be selected from the group comprising guerbet alcohols. These are liquid even at low temperatures and cause practically no skin irritations. They can advantageously be used as fatting, hyperfatting and also superfatting constituents in cosmetic compositions.

The use of guerbet alcohols in cosmetics is known per se.

Guerbet alcohols which are preferred, according to the invention are 2-butyloctanol (commercially available e.g. as Isofol®12 (Condea)) and 2-hexyldecanol (commercially available e.g. as Isofol®16, (Condea)).

Mixtures of guerbet alcohols according to the invention can also advantageously be used according to the invention, examples being mixtures of 2-butyloctanol and 2-hexyldecanol (commercially-available e.g. as Isofol®14 (Condea)).

Any desired mixtures of such oil and wax components can also advantageously be used within the framework of the present invention. Among the polyolefins, polydecenes are the preferred substances.

Fat and/or wax components which can advantageously be used according to the invention can be selected from the group comprising vegetable waxes, animal waxes, mineral waxes and petrochemical waxes. Advantageous examples are candelilla wax, carnauba wax, Japan tallow, esparto wax, cork wax, guaruma wax, rice seed oil wax, sugarcane wax, berry wax, ouricury wax, montan wax, jojoba-wax, shea butter, beeswax, shellac wax, spermaceti, lanolin (wool wax), burzel fat, ceresin, ozokerite (earth wax), paraffin waxes and microwaxes.

Other advantageous fat and/or wax components are chemically modified waxes and synthetic waxes, e.g. Syncrowax®HRC (glyceryl tribehenate) and Syncrowax®AW 1 C (C₁₈₋₃₆-fatty acid), as well as montan ester waxes, sasol waxes, hydrogenated jojoba waxes, synthetic or modified beeswaxes (e.g. Dimethicone Copolyol beeswax and/or C₃₀₋₅₀-alkyl beeswax), cetyl ricinoleates such as Tegosoft®CR, polyalkylene waxes, polyethylene glycol waxes, and also chemically modified fats such as hydrogenated plant oils (e.g. hydrogenated castor oil and/or hydrogenated coconut fatty glycerides), triglycerides such as hydrogenated soy glyceride, trihydroxystearin, fatty acids, fatty acid esters, and glycol esters such as C₂₀₋₄₀-alkyl stearate, C₂₀₋₄₀-alkylhydroxystearoyl stearate and/or glycol montanate. Certain organosilicon compounds having similar physical properties to those of said fat and/or wax components, e.g. stearoxy-trimethylsilane, are also advantageous.

According to the invention the fat and/or wax components can be used in the compositions either individually or as a mixture.

Any desired mixtures of such oil and wax components can also advantageously be used within the framework of the present invention.

The oily phase is advantageously selected from the group comprising 2-ethylhexyl isostearate, octyldodecanol, isotridecyl isononanoate, butylene glycol dicaprylate/dicaprate, 2-ethylhexyl cocoate, C₁₂₋₁₅-alkyl benzoate, caprylic/capric triglyceride and dicaprylyl ether.

Mixtures of octyldodecanol, caprylic/capric triglyceride, dicaprylyl ether, dicaprylyl carbonate and coconut glycerides, or mixtures of C₁₂₋₁₅-alkyl benzoate and 2-ethylhexyl isostearate, mixtures of C₁₂₋₁₅-alkyl benzoate and butylene glycol dicaprylate/dicaprate and mixtures of C₁₂₋₁₅-alkyl benzoate, 2-ethylhexyl isostearate and isotridecyl isononanoate are particularly advantageous.

Of the hydrocarbons, paraffin oil, cycloparaffin, squalane, squalene and hydrogenated polyisobutene or polydecene can advantageously be used within the framework of the present invention.

The oil component is also advantageously selected from the group comprising phospholipids. The following can be used according to the invention as paraffin oils which are advantageous according to the invention: Merkur®Weissoel Pharma 40 from Merkur Vaseline, Shell Ondina® 917, Shell Ondina®927, Shell Oil 4222 and Shell Ondina®0933 from Shell & DEA Oil, and Pionier® 6301 S and Pionier® 2071 (Hansen & Rosenthal).

Suitable cosmetically acceptable oil and fat components are described in Karl-Heinz Schrader, Grundlagen und Rezepturen der Kosmetika (Cosmetic Principles and Formulations), 2nd edition, Verlag Hüthig, Heidelberg, pp 319-355, which is incorporated here to the full extent by way of reference.

The content of oils, fats and waxes is at most 30, preferably 20 and particularly preferably at most 10% by weight, based on the total weight of the composition.

### Pigments

In one embodiment the compositions according to the invention contain at least one pigment. These can be colored pigments that impart color effects to the bulk product or the hair, or they can be luster effect pigments that impart luster effects to the bulk product or the hair. The color or luster effects on the hair are preferably temporary, i.e. they remain on the hair until the next wash and can be removed by washing the hair with conventional shampoos.

The pigments are in the bulk product in undissolved form and can be present in an amount of 0.01 to 25% by weight and particularly preferably of 5 to 15% by weight. The preferred particle size is 1 to 200 μm, especially 3 to 150 μm and particularly preferably 10 to 100 μm. The pigments are colorants that are practically insoluble in the application medium and they can be inorganic or organic. Mixed inorganic/organic pigments are also possible. Inorganic pigments are preferred. The advantage of inorganic pigments is their excellent light, weather and temperature resistance. The inorganic pigments can be of natural origin and prepared e.g. from chalk, ochre, umber, green earth, burnt sienna or graphite. The pigments can be white pigments, e.g. titanium dioxide or zinc oxide, black pigments, e.g. black iron oxide, colored pigments, e.g. ultramarine or red iron oxide, luster pigments, metal effect pigments, pearl luster pigments, and fluorescent or phosphorescent pigments, at least one pigment preferably being a colored, non-white pigment.

Suitable pigments are metal oxides, hydroxides and hydrated oxides, mixed phase pigments, sulfur-containing silicates, metal sulfides, complex metal cyanides, metal sulfates, chromates and molybdates, and metal themselves (bronze pigments).

Particularly suitable pigments are titanium dioxide (CI 77891), black iron oxide (CI 77499), yellow iron oxide (CI 77492), red and brown-iron oxide (CI 77491), manganese violet (CI 77742), ultramarine (sodium aluminum sulfosilicates, CI 77007, Pigment Blue 29), hydrated chromium oxide (CI 77289), Prussian blue (ferric ferrocyanide, CI 77510) and carmine (cochineal).

Particularly preferred pigments are mica-based pearl luster and colored pigments coated with a metal oxide or a metal oxychloride, such as titanium dioxide or bismuth oxychloride, and optionally other coloring substances such as iron oxides, Prussian blue, ultramarine, carmine, etc., it being possible for the color to be determined by varying the layer thickness. Such pigments are marketed e.g. under the trade marks Rona®, Colorona®, Dichrona® and Timiron® by Merck, Germany.

Examples of organic pigments are the natural pigments sepia, gamboge, bone charcoal, Cassel brown, indigo, chlorophyll and other plant pigments.

Examples of synthetic organic pigments are azo pigments, anthraquinoids, indigoids, and dioxazine, quinacridone, phthalocyanine, isoindolinone, perylene and perinone, metal complex, alkali blue and diketopyrrolopyrrole pigments.

In one embodiment the compositions according to the invention contain 0.01 to 10 and particularly preferably from 0.05 to 5% by weight of at least one particulate substance. Examples of suitable substances are those which are solid at room temperature (25° C.) and take the form of particles. Suitable substances are, for instance, silica, silicates, aluminates, aluminas, mica, salts, especially inorganic metal salts, metal oxides, e.g. titanium dioxide, minerals and polymer particles.

The particles are present in the composition in undissolved and preferably stably dispersed form and can deposit in solid form after application to the substrate surface and evaporation of the solvent.

Preferred particulate substances are silica (silica gel, silicon dioxide) and metal salts, especially inorganic metal salts, silica being particularly preferred. Examples of metal salts are alkali metal or alkaline earth metal halides, such as sodium chloride or potassium chloride, and alkali metal or alkaline earth metal sulfates, such as sodium sulfate or magnesium sulfate.

Suitable repellents are compounds that are capable of repelling particular animals, especially insects, from humans. They include e.g. 2-ethyl-1,3-hexanediol, N,N-diethyl-m-toluamide, etc.

Suitable substances with a hyperemic action, which stimulate blood circulation in the skin, are e.g. ethereal oils such as pine (Pinus montana), lavender, rosemary, juniper berry, horse chestnut extract, birch leaf extract, hayflower extract, ethyl acetate, camphor, menthol, peppermint oil, rosemary extract, eucalyptus oil, etc.

Examples of suitable substances with a keratolytic and keratoplastic action are salicylic-acid, calcium thioglycolate, thioglycolic acid and its salts, sulfur, etc. Examples of suitable antidandruff substances are sulfur, sulfur polyethylene glycol sorbitan monooleate, sulfur ricinol polyethoxylate, zinc pyrithione, aluminum pyrithione, etc.

Examples of suitable antiphlogistics, which counteract skin irritations, are allantoin, bisabolol, dragosantol, camomile-extract, panthenol, etc.

### Form of Application

In one preferred embodiment the compositions according to the invention are sprayable, e.g. as an aerosol or pump spray composition.

The compositions according to the invention can be used in various forms of application, e.g. as a lotion, a non-aerosol spray lotion, which is used with a mechanical spraying device, an aerosol spray, which is sprayed by means of a propellant, a mousse, an aerosol foam, a non-aerosol foam, which is used in combination with a suitable mechanical device for foaming the composition, a hair cream, a hair wax, a gel, a liquid gel, a sprayable gel or a foam gel.

The composition can also be used in the form of a lotion thickened with a conventional thickener.

In one embodiment the composition according to the invention is in the form of a gel, a viscous lotion or a spray gel, which is sprayed with a mechanical device, contains at least one of the abovementioned thickeners in an amount preferably of 0.05 to 10 and particularly preferably of 0.1 to 2% by weight, and has a viscosity of at least 250 mPas. The viscosity of the gel is preferably 500 to 50,000 mPas and particularly preferably from 1000 to 15,000 mPas at 25° C.

In another embodiment the composition according to the invention is in the form of an O/W emulsion, a W/O emulsion or a microemulsion and contains at least one of the abovementioned oils or waxes emulsified in water, and at least one surfactant conventionally used in cosmetics.

In one preferred embodiment the composition according to the invention is in the form of a spray product, either in combination with a mechanical pump spraying device or in combination with at least one of the abovementioned propellants. One preferred aerosol spray additionally contains propellant in an amount such that the total amount of volatile organic components does not exceed 80 and particularly 55% by weight of the composition, and is packaged in a pressurized container.

A non-aerosol hairspray is sprayed with the aid of a suitable mechanically operated spraying device. Mechanical spraying devices are understood as meaning devices which enable the composition to be sprayed without the use of a propellant. Examples of suitable mechanical spraying devices which can be used are a spray pump or an elastic container fitted with a spray valve, in which the cosmetic composition according to the invention is packaged under pressure, causing the elastic container to expand, and from which the composition is continuously released as a result of contraction of the elastic container when the spray valve is opened.

In another embodiment the composition according to the invention is in the form of a foamable product (mousse) in combination with a foaming device, and contains at least one conventional foam forming substance known for this purpose, e.g. at least one foam forming surfactant or at least one foam forming polymer. Foaming devices are understood as meaning devices which enable a liquid to be foamed with or without the use of a propellant. For example, a commercially available pump foamer or an aerosol foam head can be used as a suitable mechanical foaming device. The product is used either in combination with a mechanical pump foaming device (pump foam) or in combination with at least one propellant (aerosol foam) in an amount preferably of 1 to 20 and especially of 2 to 10% by weight. Propellants are selected e.g. from propane, butane, dimethyl ether and fluorinated hydrocarbons.

The invention thus provides a cosmetic composition, preferably for the hair, in the form of a spray product, the composition being used either in combination with a mechanical pump spraying device or in combination with at least one propellant selected from the group comprising propane, butane, dimethyl ether, fluorinated hydrocarbons and mixtures thereof.

The composition is foamed immediately before application and worked into the hair as a foam; it can then be rinsed out or left in the hair without rinsing.

One aerosol hair foam formulation which is preferred according to the invention contains
i) 0.1 to 10% by weight of at least one copolymer according to the invention,
ii) 55 to 99.8% by weight of water and alcohol,
iii) 5 to 20% by weight of a propellant,
iv) 0.1 to 5% by weight of an emulsifier, and
v) 0 to 10% by weight of other constituents,
the total amount of VOC being at most 80 and preferably 55% by weight.

Emulsifiers which can be used are any of the emulsifiers conventionally used in hair foams. Suitable emulsifiers can be non-ionic, cationic, anionic or amphoteric.

Examples of non-ionic emulsifiers (INCI nomenclature) are Laureths, e.g. Laureth-4; Ceteths, e.g. Ceteth-1, polyethylene glycol cetyl ether; Ceteareths, e.g. Ceteareth-25; polyglycol fatty acid glycerides; hydroxylated lecithin; fatty acid lactyl esters; and alkylpolyglycosides.

Examples of cationic emulsifiers are cetyldimethyl-2-hydroxyethylammonium dihydrogen phosphate, cetyltrimonium chloride, cetyltrimonium bromide, cocotrimonium methylsulfate and Quaternium-1 to x (INCI).

Anionic emulsifiers can be selected e.g. from the group comprising alkylsulfates, alkylethersulfates, alkylsulfonates, alkylarylsulfonates, alkylsuccinates, alkylsulfo-succinates, N-alkoylsarcosinates, acyltaurates, acylisethionates, alkylphosphates, alkyletherphosphates, alkylethercarboxylates and alpha-olefinsulfonates, especially the salts of alkali metals and alkaline earth metals, e.g. sodium, potassium, magnesium and calcium, and the ammonium and triethanolamine salts. The alkylethersulfates, alkyletherphosphates and alkylethercarboxylates can contain between 1 and 10 ethylene oxide or propylene oxide units, preferably 1 to 3 ethylene oxide units, per molecule.

A composition which is suitable according to the invention for styling gels can have e.g. the following formulation:
i) 0.1 to 10% by weight of at least one copolymer according to the invention,
ii) 80 to 99.85% by weight of water and alcohol,
iii) 0 to 3% by weight, preferably 0.05 to 2% by weight, of a gelling agent, and
iv) 0 to 20% by weight of other constituents,
the total amount of VOC being at most 80 and preferably 55% by weight.

Gels can be prepared using conventional gelling agents, for example in order to give the gels special rheological properties or other properties relating to application technology. Any of the gelling agents conventionally used in cosmetics can be employed. These include slightly crosslinked polyacrylic acid, e.g. Carbomer (INCI), cellulose derivatives, e.g. hydroxypropyl cellulose, hydroxyethyl cellulose and cationically modified celluloses, polysaccharides, e.g. xanthan gum, caprylic/capric triglyceride, sodium acrylate copolymers, Polyquaternium-32 (and) Paraffinum Liquidum (INCI), sodium acrylate copolymers (and) Paraffinum Liquidum (and) PPG-1 Trideceth-6, acrylamidopropyltrimonium chloride/acrylamide copolymers, Steareth-10/allyletheracrylate copolymers, Polyquaternium-37 (and) Paraffinum Liquidum (and) PPG-1 Trideceth-6, Polyquaternium-37 (and) propylene glycol dicaprate/dicaprylate (and) PPG-1 Trideceth-6, Polyquaternium-7 and Polyquaternium-44. Examples of crosslinked acrylic acid homopolymers which are suitable as gelling agents are commercially available under the name Carbopol® (Noveon). Hydrophobically modified, crosslinked polyacrylate polymers, such as Carbopol®Ultrez 21 (Noveon), are also preferred. Other examples of anionic polymers which are suitable as gelling agents are copolymers of acrylic acid and acrylamide and salts thereof, sodium salts of polyhydroxycarboxylic acids, water-soluble or water-dispersible polyesters, polyurethanes and polyureas. Particularly suitable polymers are copolymers of (meth)acrylic acid and polyetheracrylates, the polyether chain terminating with a C₈-C₃₀-alkyl radical. These include e.g. acrylate/Beheneth-25 methacrylate copolymers, which are commercially available as Aculyn® (Rohm and Haas).

In another embodiment the composition according to the invention is in the form of a hair wax, i.e. it has a waxy texture and contains at least one of the above-mentioned waxes in an amount preferably of 0.5 to 30% by weight, and optionally other water-insoluble substances. Preferred characteristics of the waxy texture are that the needle penetration index (unit of measurement 0.1 mm, test weight 100 g, test duration 5 s, test temperature 25° C.; according to DIN 51 579) is greater than or equal to 10 and particularly preferably greater than or equal to 20, and that the solidification point of the product is preferably above or equal to 30° C. and below or equal to 70° C., particularly preferably ranging from 40 to 55° C. Suitable waxes and water-insoluble substances are, in particular, emulsifiers with an HLB value below 7, silicone oils, silicone waxes, waxes (e.g. wax alcohols, wax acids, wax esters and especially natural waxes such as beeswax, carnauba wax, etc.), fatty alcohols, fatty acids, fatty acid esters or hydrophilic waxes, e.g. high-molecular polyethylene glycols having a molecular weight of 800 to 20,000 and preferably of 2000 to 10,000 g/mol.

If the cosmetic composition according to the invention, preferably for the hair, is in the form of a hair lotion, it is a substantially non-viscous or low-viscosity, flowable solution, dispersion or emulsion containing at least 10% by weight and preferably 20 to 95% by weight of a cosmetically acceptable alcohol. Alcohols which can be used in particular are the lower alcohols having 1 to 4 C atoms that are conventionally used for cosmetic purposes, e.g. ethanol and isopropanol.

If the composition according to the invention for the hair is in the form of a hair cream, it is preferably an emulsion, and either it additionally contains viscosifying ingredients in an amount of 0.1 to 10% by weight, or the required viscosity and creamy texture is built up in conventional manner by micelle formation with the aid of suitable emulsifiers, fatty acids, fatty alcohols, waxes, etc.

The copolymers according to the invention can be used in cosmetic compositions as conditioners.

The copolymers according to the invention can preferably be used in shampoo formulations as strengthening agents and/or conditioners. Preferred shampoo formulations contain
i) 0.05 to 10% by weight of at least one copolymer according to the invention,
ii) 25 to 94.950% by weight of water,
iii) 5 to 50% by weight of surfactants,
iv) 0 to 5% by weight of another conditioner, and
v) 0 to 10% by weight of other cosmetic constituents.

Any of the anionic, neutral, amphoteric or cationic surfactants conventionally used in shampoos can be used in the shampoo formulations.

Examples of suitable anionic surfactants are alkylsulfates, alkylethersulfates, alkylsulfonates, alkylarylsulfonates, alkylsuccinates, alkylsulfosuccinates, N-alkoylsarcosinates, acyltaurates, acylisethionates, alkylphosphates, alkylether-phosphates, alkylethercarboxylates and alpha-olefinsulfonates, especially the salts of alkali metals and alkaline earth metals, e.g. sodium, potassium, magnesium and calcium, and the ammonium and triethanolamine salts. The alkylethersulfates, alkyletherphosphates and alkylethercarboxylates can contain between 1 and 10 ethylene oxide or propylene oxide units, preferably 1 to 3 ethylene oxide units, per molecule.

Suitable examples are sodium laurylsulfate, ammonium laurylsulfate, sodium laurylethersulfate, ammonium laurylethersulfate, sodium laurylsarcosinate, sodium oleylsuccinate, ammonium laurylsulfosuccinate, sodium dodecylbenzenesulfonate and triethanolamine dodecylbenzenesulfonate.

Examples of suitable amphoteric surfactants are alkylbetaines, alkylamidopropyl-betaines, alkylsulfobetaines, alkyl glycinates, alkyl carboxyglycinates, alkyl ampho-acetates or amphopropionates and alkyl amphodiacetates or amphodipropionates.

It is possible e.g. to use cocodimethylsulfopropylbetaine, laurylbetaine, cocamido-propylbetaine or sodium cocamphopropionate.

Examples of suitable non-ionic surfactants are the reaction products of aliphatic alcohols or alkylphenols having 6 to 20 C atoms in the alkyl chain, which can be linear or branched, with ethylene oxide and/or propylene oxide. The amount of alkylene oxide is approx. 6 to 60 mol per mol of alcohol. Alkylamine oxides, mono- or dialkylalkanolamides, fatty acid esters of polyethylene glycols, alkylpolyglycosides or sorbitanetheresters are also suitable.

The shampoo formulations can also contain conventional cationic surfactants such as quaternary ammonium compounds, e.g. cetyltrimethylammonium chloride.

In the shampoo formulations, conventional conditioners can be used in combination with the copolymers according to the invention in order to achieve specific effects. Said conditioners include e.g. the abovementioned cationic polymers having the INCI name Polyquaternium, especially copolymers of vinylpyrrolidone/N-vinylimidazolium salts (Luviquat®FC, Luviquat®HM, Luviquat®MS, Luviquat®Care, Luviquat®Ultracare, Luviquat®Supreme), N-vinylpyrrolidone/dimethylaminoethyl methacrylate copolymers quaternized with diethyl sulfate; (Luviquat®PQ 11), copolymers of N-vinylcaprolactam/N-vinylpyrrolidone/N-vinylimidazolium salts (Luviquat®Hold), cationic cellulose derivatives (Polyquaternium-4 and -10) and acrylamide copolymers (Polyquaternium-7). It is also possible to use protein hydrolyzates and conditioners based on silicone compounds, e.g. polyalkylsiloxanes, polyarylsiloxanes, polyarylalkylsiloxanes, polyethersiloxanes or silicone resins. Other suitable silicone compounds are Dimethicone Copolyols (CTFA) and amino-functional silicone compounds; such as Amodimethicones (CTFA). Cationic guar derivatives, such as guar hydroxy-propyltrimonium chloride (INCI), can also be used.

### Methods of Measurement

Determination of the K Value

The K values are measured according to Fikentscher, Cellulosechemie (Cellulose Chemistry), vol. 13, pp 58 to 64 (1932), at 25° C. in ethanol or N-methylpyrrolidone (NMP) solution and are a measure of the molecular-weight. The ethanol or NMP solutions of the polymers each contain 1 g of polymer A in 100 ml of solution.

If the polymers are in the form of aqueous dispersions, amounts of the dispersion corresponding to its polymer content are made up to 100 ml with ethanol to give a concentration of 1 g in 100 ml.

The K value is measured in a Schott type M Ic micro-Ubbelohde capillary.

### Determination of the Droplet Size Distribution (DSD) by Means of Malvern® Light-Scattering Analysis

The droplet size distribution was determined with the “Malvern®Master Sizer X” particle size measuring system for detecting liquid aerosols (Malvern Instruments Inc., Southborough Mass., USA).

### Principle of Measurement:

The measuring system is based on the method of laser light diffraction at the particle, which, apart from spray analysis (aerosols, pump sprays), is also suitable for the size determination of solids, suspensions and emulsions in the range from 0.1 μm to 2000 μm.

A droplet is illuminated by a laser. Part of the incident laser light is scattered at each droplet. This light is collected at a multielement detector and the corresponding light energy distribution is determined. The evaluation software uses these data to calculate the corresponding particle distribution.

### Procedure:

The aerosols were sprayed in at a distance of 29.5 cm from the laser beam. The spray cone entered at right angles to the laser beam.

Before each measurement the aerosol cans were fixed to a firmly installed holding device so that all the aerosols to be tested Were measured at exactly the same distance.

Before the actual particle measurement a “background measurement” was made to eliminate the effects of dust and other contaminants in the measurement area.

The aerosol was then sprayed into the test space. The total particle volume was detected over a test duration of 2 s and evaluated.

### Evaluation:

The evaluation comprises a tabular representation over 32 class widths from 0.5 μm to 2000 μm and also a graphical representation of the particle size distribution.

As the spray tests involve an approximately uniform distribution, the mean diameter D(v,0.5) is given. This numerical value indicates that 50% of the total particle volume measured is below this value.

In the case of easily sprayable aerosol systems in the cosmetic sector, this value ranges from 30 μm to 80 μm, depending on the polymer content, valve and spray head geometry, solvent ratio and amounts of propellant gas.

### Determination of the Strengthening Action (Flexural Rigidity):

In addition to, subjective assessment (hand test), the strengthening action of polymeric film-forming agents was also measured physically by measurement of the flexural rigidity of thin swatches of hair (each weighing approx. 3 g and 24-cm in length). This was done by immersing the weighed, dry swatches in a 3.0% by weight polymer solution (solvent: ethanol/water 55:45 w/w), a uniform wetting of the swatches and distribution of the polymer solution being assured by immersing and removing them three times and then squeezing them between filter paper. The excess solution of film-forming agent was then wiped off between thumb and index finger and the swatches were shaped by hand to give them a round cross-section. They were dried overnight in a climate-controlled room at 20° C. and 65% relative humidity. The tests were performed on a tensile tester in the climate-controlled room at 20° C. and 65% relative humidity. The swatch was placed symmetrically at the ends of two cylindrical rollers of the sample holder. Precisely in the middle, the swatch was then bent by approx. 40 mm from above with a rounded die (breaking of the polymer film). The force required (Fmax) was determined with a load cell, (50 N). A measured value represents the arithmetic mean of the individual measurements on 5 to 10 identically treated swatches. The values determined were compared with those of a commercially available reference, polymer (Amphomer®LV-71) and given in %.

### Determination of the Ease of Rinsing:

A swatch treated with polymer analogously to the determination of strengthening action was washed for approx. 15 seconds in Texapon®NSO solution at approx. 37° C. (6 ml of Texapon®NSO (28%) in 1 l of warm water) by being immersed 5 times and squeezed. The swatch was then rinsed until the rinse water was clear, and treated again in the same way. It was then squeezed thoroughly on filter paper and dried overnight. The dry swatch was placed in rollers and examined for residues.

### Determination of the Curl Retention

### Basic Formulation (Aerosol Hairspray):

5% by weight of active substance: test polymer (100% neutralized with AMP)
15% by weight of ethanol
40% by weight of water
40% by weight of dimethyl ether

The curl retention was determined using swatches weighing approx. 2 g and 15.5 cm in length, taken from fair Caucasian human hair.

### Treatment of the Swatches:

The swatches were washed twice with aqueous Texapon®NSO solution. They were then rinsed with warm water until foaming was no longer detectable, rinsed again with demineralized water, combed and placed on filter paper to dry.

A water-wave is produced by swelling the swatches for 15 minutes in a solution of ethanol and water (1:1).

The swatch was carefully combed before preparation of the curl. It was fixed to a Plexiglas rod with a rubber band and then combed and wound into a spiral. The curl was firmly fixed with a cotton cloth and rubber band and dried overnight at 70° C. After cooling, the curl retention-swatches were carefully opened and slipped off the Plexiglas rod without deforming the water-wave. 1.8 g of the aerosol hairspray prepared as above were uniformly sprayed onto the curl from a distance of 15 cm while the curl was being steadily rotated. The curls were dried horizontally for 1 h at room temperature. After drying, they were fixed in a holder. Using a ruler, the initial length of the curls was recorded and the increase in length was followed during storage in a humidified climate. After storage for 5 h at 25° C. and 90%, RH in the climate-controlled chamber, the final length of the curl was recorded and the curl retention calculated according to the following equation:

$$curl retention in \% = \frac{L - {L}_{t}}{L - {L}_{o}} * 100$$

L=length of hairs (15.5 cm)
L₀=length of hair curl after drying
Lₜ=length of hair curl after-climate treatment

The curl retention was given as the mean of the 5 individual measurements.

### Determination of the Stickiness

Firstly, a clear, 20% by weight ethanolic or ethanolic-aqueous solution of the polymer to be characterized was prepared. It was necessary to neutralize the polymer in some cases in order to obtain a clear solution. Using a blade (120 μm slot width), a film of the polymer was applied to a glass plate from the ethanolic or ethanolic-aqueous solution. This rectangular glass plate had a length of approx. 20 cm and a width of approx. 6.5 cm. The polymer film applied thereto had a length of approx. 16 to 18 cm and a width of approx. 5.5 cm in each case.

The film was then dried in the air for approx. 10 hours and subsequently stored for a further 12 hours in a climate-controlled cabinet at 20° C. and 80% relative humidity.

In the cabinet, under these conditions, a plastic-carbon band (e.g. Pelikan®2060, 50 mm wide) attached to a round rubber stamp: (diameter 400 mm, Shore A hardness 60±5) was then pressed onto the polymer film for 10 seconds with a force of approx. 250N.

The amount of black pigment adhering to the polymer film after removal of the stamp corresponds to the stickiness of the film. The blacking of the film was assessed visually. The assessment scale ranges from 0 to 5, 0 signifying non-sticky and 5 very sticky.

### Determination of the Appearance of the Aerosol Formulation

A transparent glass aerosol container was filled with a composition consisting of 5% by weight of the respective polymer neutralized with AMP, 40% by weight of DME, 15% by weight of ethanol and 40% by weight of water. The clarity of the resulting liquid/propellant gas mixture was then assessed visually.

The Examples which follow will illustrate the subject of the invention in greater detail.

### Abbreviations Used:

EMA: ethyl methacrylate (Compound a)
MMA: methyl methacrylate (Compound a)
MA: methacrylic acid (Compound b)
AA: acrylic acid (Compound c)

### NtBAEMA: N-(tert-butyl)aminoethyl(meth)acrylate (Compound d)

DMAPMAM: N-[3-(dimethylamino)propyl]methacrylamide (Compound d)
QVI: N-vinylimidazole quaternized with dimethyl sulfate before polymerization (Compound d)
Quat-311: N,N-dimethylaminoethyl methacrylate quaternized with methyl chloride before polymerization (Compound d)
Belsil® DMC 6031: silicone compound containing polyalkylene oxide (Compound f)

### CE1

### Comparative Example 1

Preparation of the copolymers
Example: Preparation of Polymer 25
MMA/MA/AA/Quat 311//Belsil ® DMC 6031 66/12/12/10 // 0.2

| | | |
|---|---|---|
| Initial ingredients: | 190 g | demineralized water |
| | 150 g | isopropanol |
| | 35 g | Addition 1 |
| | 5 g | Addition 2 |
| | 0.9 g | Belsil ® DMC 6031 |
| Addition 1: | 257.4 g | methyl methacrylate |
| | 80 g | Quat 311 (50%) |
| | 46.8 g | MA |
| | 46.8 g | AA |
| | 275 g | isopropanol |
| Addition 2: | 75 g | isopropanol |
| | 7.8 g | Wako ® V59 |
| Addition 3: | 230 g | isopropanol |
| | 4 g | tert-butyl perpivalate |
| Addition 4: | 55 g | AMP |
| | 55 g | water |
| Addition 5: | 390 g | ethanol |

The initial ingredients were heated to approx. 75° C. under a nitrogen atmosphere. Addition 1 was introduced over 4 hours and Addition 2 over 5 hours. The polymerization solution was stirred for a further 2 hours at 78° C. Addition 3 was then introduced over 30 minutes, this being followed by stirring at 80° C. for a further 4 hours. Addition 4 was then introduced and the mixture was homogenized for 30 minutes. Isopropanol was distilled off at an oil bath temperature of 120° C. until the reaction solution reached a temperature of approx. 85° C. This was followed by steam distillation until the temperature of the reaction solution was approx. 100° C. After cooling to 40° C., Addition 5 was metered in and the mixture was stirred until a homogeneous phase was obtained. The phase was then diluted with water to a solids content of 30% by weight.

Polymers 1-27 in the Table below were polymerized analogously to the procedure described above.

TABLE
Survey of copolymers according to the invention
The amounts given in the following Table for the individual components a) to e) are in
% by weight of the total amount of monomers used for the polymerization.
The amounts given for the silicone compound f) containing polyalkylene oxide, i.e.
Belsil ®, are in % by weight, based on 100% by weight of the total amount of
components a) to e).

| | | | | | | | | | Belsil ® | |
|---|---|---|---|---|---|---|---|---|---|---|
| | | | | | NtBA | DMAP | | | DMC | |
| Polymer | EMA | MMA | MA | AA | EMA | MAM | QVI | Quat-311 | 6031 | K value |
| CE1 | 80 | — | 10 | 10 | — | — | — | — | — | 42.2 |
| 1* | 78 | — | 10 | 10 | — | 2 | — | — | — | 40.2 |
| 2* | 79 | — | 10 | 10 | — | 1 | — | — | — | 37.8 |
| 3 | 79 | — | 10 | 10 | — | 1 | — | — | 1 | 36.0 |
| 4* | 78 | — | 10 | 10 | — | 2 | — | — | | 34.9 |
| 5 | — | 77.5 | 11 | 11 | — | — | — | — | 0.5 | 33.8 |
| 6 | — | 73.8 | 13 | 13 | — | — | — | — | 0.2 | 33.8 |
| 7 | — | 79 | 10 | 10 | — | 1 | — | — | — | 38.5 |
| 8 | — | 78 | 10 | 10 | — | 2 | — | — | — | 37.6 |
| 9 | — | 79 | 10 | 10 | — | 1 | — | — | 1 | 38.1 |
| 10 | — | 78 | 10 | 10 | — | 2 | — | — | — | 35.1 |
| 11 | 78 | — | 10 | 10 | — | 2 | — | — | 0.2 | 34.7 |
| 12 | 75* | — | 19 | 05 | 1 | — | — | — | — | 34.7 |
| 13 | 78 | — | 10 | 10 | — | — | 2 | — | 0.3 | 33.4 |
| 14 | 77 | — | 10 | 10 | — | — | — | 3 | 0.3 | 34.1 |
| 15 | — | 75 | 12 | 12 | — | — | 1 | — | 0.2 | 31.9 |
| 16 | — | 75 | 11 | 11 | — | — | 3 | — | 0.1 | 33.6 |
| 17 | — | 72 | 13 | 12 | — | — | — | 3 | 0.2 | 34.9 |
| 18 | — | 72 | 12 | 12 | — | — | — | 4 | 0.2 | 33.4 |
| 19** | — | 70 | 12 | 12 | — | — | — | 6 | 0.2 | 34.2 |
| 20** | — | 70 | 11 | 11 | — | — | — | 8 | 0.2 | 33.3 |
| 21** | — | 68 | 12 | 12 | — | — | — | 8 | — | 31.1 |
| 22** | — | 68 | 12 | 12 | — | — | — | 8 | 0.2 | 32.5 |
| 23** | — | 66 | 13 | 13 | — | — | — | 8 | 0.3 | 31.9 |
| 24** | — | 64 | 14 | 14 | — | — | — | 8 | 0.2 | 33.1 |
| 25** | — | 66 | 12 | 12 | — | — | — | 10 | 0.2 | 34.1 |
| 26** | — | 66 | 12 | 10 | — | — | — | 12 | 0.1 | 33.1 |
| 27** | — | 65 | 10 | 10 | — | — | — | 15 | — | 32.9 |

*Examples not according to the invention
**6 g of Wako ® V59 were used to prepare these polymers.

### APPLICATION EXAMPLES

In Examples 1 to 7 below, the respective polymers according to the invention were used as a 30% by weight solution in an ethanol/water mixture. The percentages accordingly relate to the amount of this solution used. [%] denotes % by weight.

1) VOC-80 aerosol hairspray

| | [%] |
|---|---|
| Polymer 3 | 12.00 |
| Water | 11.60 |
| Ethanol | 36.40 |
| If the pH is <7, it is adjusted to 7-7.5 with AMP. | |
| Dimethyl ether | 40.00 |
| other optional additives: | silicone, perfume, defoamer, etc. |

The Example can be repeated with the polymers of Examples 5 to 11 and 13 to 27. A VOC-80 aerosol hairspray with good properties is obtained in each case.

2) VOC-80 aerosol hairspray

| | [%] |
|---|---|
| Polymer 3 | 10.00 |
| Luvimer ® 100P | 1.0 |
| Water | 12.00 |
| Ethanol | 36.65 |
| AMP (95%) | 0.35 |
| Dimethyl ether | 40.00 |
| other optional additives: | silicone, perfume, defoamer, etc. |

The Example can be repeated with the polymers of Examples 5 to 11 and 13 to 27. A VOC-80 aerosol hairspray with good properties is obtained in each case.

3) VOC-55 aerosol hairspray

| | [%] |
|---|---|
| Polymer 3 | 15.00 |
| Water | 34.50 |
| Ethanol | 15.50 |
| Dimethyl ether | 35.00 |
| If the pH is <7, it is adjusted to 7-7.5 with AMP. | |
| other optional additives: | silicone, perfume, defoamer, etc. |

The Example can be repeated with the polymers of Examples 5 to 11 and 13 to 27. A VOC-55 aerosol hairspray with good properties is obtained in each case.

4) VOC-55 aerosol hairspray

| | [%] |
|---|---|
| Polymer 3 | 12.00 |
| Luviset ® P.U.R. | 3.00 (30% by weight in water/ethanol 6/1 w/w) |
| Water | 34.50 |
| Ethanol | 15.50 |
| Dimethyl ether | 35.00 |
| other optional additives: | silicone, perfume, defoamer, etc. |

The Example can be repeated with the polymers of Examples 5 to 11 and 13 to 27. A VOC-55 aerosol hairspray with good properties is obtained in each case.

5) VOC-55 aerosol hairspray

| | [%] |
|---|---|
| Polymer 3 | 12.00 |
| Luvimer ® Pro55 | 3.00 (30% by weight aqueous dispersion) |
| Water | 33.30 |
| Ethanol | 11.40 |
| Aminomethylpropanol (95%) | 0.30 |
| Dimethyl ether | 40.00 |
| other optional additives: | silicone, perfume, defoamer, etc. |

The Example can be repeated with the polymers of Examples 5 to 11 and 13 to 27. A VOC-55 aerosol hairspray with good properties is obtained in each case.

6) VOC-55 pump spray

| | [%] |
|---|---|
| Polymer 3 | 15.00 |
| Water | 34.50 |
| Ethanol | 50.50 |
| If the pH is <7, it is adjusted to 7-7.5 with AMP. | |
| other optional additives: | silicone, perfume, defoamer, etc. |

The Example can be repeated with the polymers of Examples 5 to 11 and 13 to 27. A VOC-55 pump spray with good properties is obtain in each case.

7) VOC-<10 pump spray

| | [%] |
|---|---|
| Polymer 3 | 12.00 |
| Luviset ® Clear | 5.00 (20% by weight aqueous solution) |
| Water | 83.00 |

If the pH is <7, it is adjusted to 7-7.5 with AMP.

| | |
|---|---|
| other optional additives: | silicone, perfume, defoamer, etc. |

The Example can be repeated with the polymers of Examples 5 to 11 and 13 to 27. A pump spray with good properties is obtained in each case.

1. A cosmetic composition comprising a copolymer obtainable by the copolymerization of
a) at least one methacrylic acid ester selected from the group consisting of methyl methacrylate, ethyl methacrylate, n-propyl methacrylate, i-propyl methacrylate and mixtures thereof,
b) methacrylic acid,
c) acrylic acid,
d) at least one cationic or cationogenic, olefinically unsaturated compound containing amino groups and polymerizable by free radical polymerization, and
e) optionally other olefinically unsaturated compounds polymerizable by free radical polymerization,
the copolymerization being carried out in the presence of a silicone f) containing polyalkylene oxide if no compound d) is copolymerized, with the proviso that the copolymerization is carried out in the presence of at least one silicone f) containing polyalkylene oxide if a) is or contains ethyl methacrylate.

2. The cosmetic composition as claimed in claim 1 wherein a) is selected from methyl methacrylate, ethyl methacrylate and mixtures thereof.

3. The cosmetic composition as claimed in claim 1 wherein d) is selected from the consisting of
d1) esters of α,β-ethylenically unsaturated monocarboxylic and dicarboxylic acids with amino alcohols which can be monoalkylated or dialkylated on the amine nitrogen,
d2) amides of α,β-ethylenically unsaturated monocarboxylic and dicarboxylic acids with diamines having at least one primary or secondary amino group,
d3) N,N-diallylamine and derivatives thereof,
d4) vinyl- and allyl-substituted nitrogen heterocycles,
d5) vinyl- and allyl-substituted heteroaromatic compounds, and
d6) mixtures thereof.

4. The cosmetic composition as claimed in claim 1 wherein the compound d) is used for the polymerization in a form quaternized to the extent of at least 50 mol %.

5. The cosmetic composition as claimed in claim 1 wherein the copolymer is obtainable by the copolymerization of
a) 50-85% by weight of methyl methacrylate,
b) 5-30% by weight of methacrylic acid,
c) 5-30% by weight of acrylic acid,
d) 0.1-20% by weight of at least one olefinically unsaturated compound containing amino groups and polymerizable by free radical polymerization, and
e) 0-10% by weight of other olefinically unsaturated compounds polymerizable by free radical polymerization,
the amounts of components a) to e) adding up to 100% by weight.

6. The cosmetic composition as claimed in claim 1 wherein the copolymer is obtainable by the copolymerization of
a) 50-85% by weight of methyl methacrylate and/or ethyl methacrylate,
b) 5-30% by weight of methacrylic acid,
c) 5-30% by weight of acrylic acid,
d) 0.1-20% by weight of at least one olefinically unsaturated compound containing amino groups and polymerizable by free radical polymerization, and
e) 0-10% by weight of other olefinically unsaturated compounds polymerizable by free radical polymerization,
the amounts of components a) to e) adding up to 100% by weight and the copolymerization being carried out in the presence of 0.1-3% by weight, based on the sum of components a) to e), of at least one silicone f) containing polyalkylene oxide.

7. The cosmetic composition as claimed in claim 1 wherein the copolymer is obtainable by the copolymerization of
a) 50-80% by weight of methyl methacrylate,
b) 8-20% by weight of methacrylic acid,
c) 5-15% by weight of acrylic acid,
d) 0.5-15% by weight of at least one olefinically unsaturated compound containing amino groups and polymerizable by free radical polymerization, and
e) 0-10% by weight of other olefinically unsaturated compounds polymerizable by free radical polymerization,
the amounts of components a) to e) adding up to 100% by weight and the copolymerization being carried out in the presence of at least one silicone f) containing polyalkylene oxide.

8. The cosmetic composition as claimed in claim 1 wherein the compound d) is selected from the group consisting of N-[3-(dimethylamino)propyl]-(meth)acrylamide, N,N-dimethylaminoethyl(meth)acrylate, N-vinylimidazole and mixtures thereof.

9. The cosmetic composition as claimed in claim 1 claim 1 wherein the copolymer is obtainable by the copolymerization of
a) 50-80% by weight of methyl methacrylate and/or ethyl methacrylate,
b) 5-30% by weight of methacrylic acid,
c) 5-30% by weight of acrylic acid,
d) 0% by weight of compound d), and
e) 0-10% by weight of other olefinically unsaturated compounds polymerizable by free radical polymerization,
the amounts of components a) to e) adding up to 100% by weight and the copolymerization being carried out in the presence of at least one silicone f) containing polyalkylene oxide.

10. The cosmetic composition as claimed in claim 1 wherein the amount of silicone f) containing polyalkylene oxide is 0.1-1% by weight, based on the sum of components a) to e), if the copolymerization is carried out in the presence of at least one silicone f) containing polyalkylene oxide.

11. The cosmetic composition as claimed in claim 1 which also contains water and at least one cosmetically acceptable carrier B) selected from
i. water-miscible organic solvents,
ii. oils, fats and waxes,
iii. esters of C₆-C₃₀-monocarboxylic acids with mono-, di- or trihydric alcohols, which differ from ii),
iv. saturated acyclic and cyclic hydrocarbons,
v. fatty acids,
vi. fatty alcohols,
vii. propellants (propellant gases) and
viii. mixtures thereof.

12. The cosmetic composition as claimed in claim 1 in the form of a spray product which is used either in combination with a mechanical pump spraying device or in combination with at least one propellant selected from the group consisting of propane, butane, dimethyl ether, fluorinated hydrocarbons and mixtures thereof.

13. The cosmetic composition as claimed in claim 1 wherein the VOC content is at most 80 by weight of the composition.

14. The cosmetic composition as claimed in claim 1 wherein the pH of the composition ranges from 6.5 to 8.5.

15. A copolymer as defined in claim 1.

16. The process for the preparation of a copolymer as claimed in claim 15, wherein the copolymerization is carried out as a solution polymerization
in a solvent comprising or consisting of a mixture of at most 99% by weight of alcohol and at least 1% by weight of water,
at 65-120° C.,
in the presence of at least one organic initiator,
at least 30 mol % of the acid groups of the copolymer being neutralized with at least one amine containing hydroxyl groups when the residual monomer content is at most 5, based on the polymerization solution, and
at least 90% of the organic solvent being removed by steam distillation when the residual monomer content is at most 0.2% by weight, based on the polymerization solution.

17. The process as claimed in claim 16 wherein the amine containing hydroxyl groups is selected from the group consisting of N,N-dimethylethanolamine, N-methyldiethanolamine, triethanolamine, 2-amino-2-methylpropanol and mixtures thereof.

18. The cosmetic composition as claimed in claim 2 wherein d) is selected from the group consisting of
d1) esters of α,β-ethylenically unsaturated monocarboxylic and dicarboxylic acids with amino alcohols which can be monoalkylated or dialkylated on the amine nitrogen,
d2) amides of α,β-ethylenically unsaturated monocarboxylic and dicarboxylic acids with diamines having at least one primary or secondary amino group,
d3) N,N-diallylamine and derivatives thereof,
d4) vinyl- and allyl-substituted nitrogen heterocycles,
d5) vinyl- and allyl-substituted heteroaromatic compounds, and
d6) mixtures thereof.

19. The cosmetic composition as claimed in claim 2 wherein the compound d) is used for the polymerization in a form quaternized to the extent of at least 50 mol %.

20. The cosmetic composition as claimed in claim 3 wherein the compound d) is used for the polymerization in a form quaternized to the extent of at least 50 mol %.
